(19) United States
(12) Patent Application Publication
(10) Pub. No.: US 2025/0302751 A1
(43) Pub. Date: Oct. 2, 2025

(54) FORMULATIONS FOR ADMINISTERING LAAM, norLAAM AND dinorLAAM AND METHODS OF THEIR USE TO TREAT OPIOID USE DISORDER

(71) Applicants: VIRGINIA COMMONWEALTH UNIVERSITY, Richmond, VA (US); UNIVERSITY OF CONNECTICUT, Stoars (CT)

(72) Inventors: Qingguo XU, GLEN ALLEN, VA (US); Charles O'KEEFE, MIDLOTHIAN, VA (US); Andre O'Reilly BERINGHS, WILLINGTON, CT (US); Aji Alex Moothedathu RAYNOLD, RICHMOND, VA (US); Hadi SUDARJAT, RICHMOND, VA (US); Zixuan ZHEN, RICHMOND, VA (US)

(21) Appl. No.: 18/864,588

(22) PCT Filed: May 19, 2023
(86) PCT No.: PCT/US2023/067216

§ 371 Date: Nov. 11, 2024

Related U.S. Application Data
(60) Provisional application No. 63/343,811, filed on May 19, 2022.
(60) Provisional application No. 63/345,126, filed on May 24, 2022.

(51) Int. Cl. A61K 9/20; A61K 9/16; A61K 9/50; A61K 9/70; A61K 31/22; A61P 25/36

(52) U.S. Cl. CPC A61K 9/2054; A61K 9/1641; A61K 9/1652; A61K 9/2031; A61K 9/50; A61K 9/7007; A61K 31/22; A61P 25/36

(57) ABSTRACT

Rapid-release formulations for administering Levo-alpha-acetylmethadol (LAAM), norLAAM and dinorLAMM and, optionally, magnesium, are provided. The formulations include solid i) core-shell oral dosage forms delivered in capsules or tablets, and ii) electrospun nano/microfiber buccal film dosage forms. Methods of the use of the formulations to treat opioid use disorder (OUD) and pain are also provided.

### CROSS-REFERENCE TO RELATED APPLICATIONS

This application claims benefit of U.S. Provisional patent applications 63/343,811 filed May 19, 2022, and 63/345,126 filed May 24, 2022.

### STATEMENT OF FEDERALLY SPONSORED RESEARCH AND DEVELOPMENT

This invention was made with government support under grant number(s) DA048768 awarded by the National Institutes of Health (NIH). The government has certain rights in the invention.

### BACKGROUND OF THE INVENTION

### Technical Field

The invention generally relates to improved compositions and methods for administering levo-alpha-acetylmethadol (LAAM), norLAAM and dinorLAMM, and optionally Mg, for the treatment of opioid use disorder (OUD) or pain. In particular, the invention provides rapid release i) core-shell pellets for oral drug (i.e., LAAM, norLAAM or dinorLAAM) delivery via capsule or tablet, ii) core-shell tablets for oral drug (i.e., LAAM, norLAAM or dinorLAAM) delivery, and ii) electrospun nano/microfiber films for buccal administration to deliver LAAM, norLAAM or dinorLAAM.

### Description of Related Art

Since 1999, approximately 700,000 people have died from opioid-related overdoses, a significant public health concern. The surge in Opioid Use Disorder (OUD) related fatalities has prompted efforts to reduce disease progression. Although OUD therapies such as methadone or buprenorphine have significantly improved treatment outcomes, they are still not fulfilling the needs of many patients. For example, they require daily administration at treatment clinics and patients encounter difficulties meeting this demand, resulting in poor patient compliance and retention. Therapies with greater efficacy and longer duration could significantly enhance treatment outcomes in response to these difficulties. Levo-alpha-acetylmethadol (LAAM) is an example of a treatment that offers numerous behavioral and clinical advantages for patients who are not sufficiently managed by buprenorphine, naltrexone or methadone. Compared to available OUD treatments, LAAM has a higher systemic circulation half-life, which reduces the dosing frequency to 3 times per week and improves patient compliance. LAAM has been shown to be more effective than methadone in reducing illegal opioid use and drug-related imprisonment and improving patient retention and satisfaction. LAAM was approved for use in the U.S. in 1993 but unfortunately, its use is associated with the risk of life-threatening QTc interval prolongation and potential torsade de pointes (TdP). Buprenorphine and naltrexone do not affect cardiac conduction, but methadone does. While methadone also can cause QTc prolongation and TdP, the FDA only required ECG screening for methadone if the patient has cardiac conduction abnormalities or is taking medications that affect cardiac conduction. In contrast, the FDA required ECG testing and ongoing monitoring when LAAM was used. Many clinics were reluctant or unable to perform regular ECG screenings, causing the use of LAAM to drop drastically. In 2001, LAAM was removed from the European market due to reports of life-threatening ventricular rhythm disorders and was subsequently withdrawn from the US market in 2003. LAAM's absence from the clinic denotes a significant loss to addiction treatment.

There is a pressing need to develop new methods of treating OUD. In particular, it would be highly beneficial to reintroduce LAAM, norLAAM and/or dinorLAAM as a treatment using improved formulations which reduce the optimal therapeutic dose required and thus reduce adverse side effects.

### SUMMARY OF THE INVENTION

Other features and advantages of the present invention will be set forth in the description of invention that follows, and in part will be apparent from the description or may be learned by practice of the invention. The invention will be realized and attained by the compositions and methods particularly pointed out in the written description and claims hereof.

Disclosed herein are novel levo-alpha-acetylmethadol (LAAM) or metabolic products thereof (norLAAM, dinorLAAM) rapid release dosage forms as effective medications (medicaments) to treat opioid use disorders and/or pain. The novel LAAM, norLAAM and dinorLAAM formulations provide a substantial improvement to OUD treatment. The formulations are used in an approximately 2-3 times per week dosing regimen due to their longer systemic half-lives. This improves treatment by reducing the frequency of clinical visits, and thus promoting patient compliance, while minimizing costs. The novel dosage forms help medical providers improve treatment outcomes for patients who have not adequately responded to prior art therapies. LAAM's return to the market provides an additional effective medication to treat OUD, significantly reducing the alarming number of opioid-related overdoses and deaths. In some aspects, the formulations also include magnesium (Mg), typically as a Mg salt, to prevent QTc interval prolongation and potential torsade de pointes (TdP).

In one aspect, pellets comprising a solid core surrounded by a rapid-release polymer matrix (shell) comprising LAAM (or in some aspects, a metabolic breakdown product thereof) are provided. For delivery, the drug-loaded pellets are typically embedded in tablets or encased in capsules, making it possible to orally administer different dose strengths by loading different amounts and/or strengths of pellets into the tablet or capsule. When exposed to the aqueous physiological environment, the capsule or tablet and the polymer matrix of the pellets within dissolves and releases the drug rapidly. For example, total release of the dose is realized in about 10-60 minutes, or less. In another aspect, tablets comprising a solid core surrounded by a rapid-release polymer matrix (shell) comprising LAAM (or in some aspects, a metabolic breakdown product thereof) are provided. In yet other aspects, films formed from electrospun fibers comprising LAAM (or a metabolic breakdown product thereof) are provided for buccal administration. The films, which comprise an impermeable backing, have mucoadhesive properties that permit adhesion to the buccal mucosa, and the rapid unidirectional transport of LAAM, norLAAM or dinorLAAM out of the films and across the buccal mucosa, with complete release of the dose occurring within about 10-60 minutes or less. Any of these dosage forms may optionally also contain Mg, usually a Mg salt.

It is an object of this invention to provide a pellet or tablet comprising a solid core, and a solid matrix surrounding the solid core, wherein the solid matrix comprises at least one layer comprising a water-soluble polymer and levo-alpha-acetylmethadol (LAAM) or a physiologically active metabolite thereof. In some aspects, the solid core is or comprises microcrystalline cellulose. In other aspects, the water-soluble polymer is or comprises a polyethylene glycol-polyvinyl alcohol graft copolymer. In further aspects, the polyethylene glycol-polyvinyl alcohol graft copolymer and the LAAM or physiologically active metabolite thereof are present at ratio of 1:2. In additional aspects, the solid matrix comprises 2% (w/w) of LAAM or the physiologically active metabolite thereof. In some aspects, the solid matrix comprises a plurality of layers. In other aspects, the physiologically active metabolite is norLAAM or dinorLAMM. In further aspects, the solid core is or comprises Mg or a salt thereof.

Also provided is a water-soluble film comprising electrospun fibers comprising at least one mucoadhesive polymer and LAAM or a physiologically active metabolite thereof and, optionally Mg or a salt thereof. In some aspects, the at least one mucoadhesive polymer is a copolymer comprising ethyl acrylate, methyl methacrylate and methacrylic acid ester with quaternary ammonium groups. In other aspects, the water-soluble film comprises a backing layer that is impermeable to the LAAM or a physiologically active metabolite thereof. In certain aspects, the backing layer comprises a copolymer comprising poly(ethyl acrylate, methyl methacrylate) and polyethylene glycol stearyl ether. In additional aspects, the electrospun fibers further comprise one or more of a penetration enhancer, a plasticizer, a sweetener and an antioxidant. In yet further aspects, the physiologically active metabolite is norLAAM or dinorLAMM. In additional aspects, the comprising the water-soluble film comprises Mg or a salt thereof.

Also provided is a medicament for the treatment of OUD or pain, comprising the tablet of claim any of claims 1-8, i.e., a pellet or tablet comprising a solid core, and a solid matrix surrounding the solid core, wherein the solid matrix comprises at least one layer comprising a water-soluble polymer and levo-alpha-acetylmethadol (LAAM) or a physiologically active metabolite thereof. In some aspects, the solid core is or comprises microcrystalline cellulose. In other aspects, the water-soluble polymer is or comprises a polyethylene glycol-polyvinyl alcohol graft copolymer. In further aspects, the polyethylene glycol-polyvinyl alcohol graft copolymer and the LAAM or physiologically active metabolite thereof are present at ratio of 1:2. In additional aspects, the solid matrix comprises 2% (w/w) of LAAM or the physiologically active metabolite thereof. In some aspects, the solid matrix comprises a plurality of layers. In other aspects, the physiologically active metabolite is norLAAM or dinorLAMM. In further aspects, the solid core is or comprises Mg or a salt thereof.

Also provided is a medicament for the treatment of OUD or pain, comprising
a capsule containing a plurality of pellets of or a tablet comprising a plurality of pellets comprising a solid core, and a solid matrix surrounding the solid core, wherein the solid matrix comprises at least one layer comprising a water-soluble polymer and levo-alpha-acetylmethadol (LAAM) or a physiologically active metabolite thereof. In some aspects, the solid core is or comprises microcrystalline cellulose. In other aspects, the water-soluble polymer is or comprises a polyethylene glycol-polyvinyl alcohol graft copolymer. In further aspects, the polyethylene glycol-polyvinyl alcohol graft copolymer and the LAAM or physiologically active metabolite thereof are present at ratio of 1:2. In additional aspects, the solid matrix comprises 2% (w/w) of LAAM or the physiologically active metabolite thereof. In some aspects, the solid matrix comprises a plurality of layers. In other aspects, the physiologically active metabolite is norLAAM or dinorLAMM. In further aspects, the solid core is or comprises Mg or a salt thereof.

Also provided is a medicament for the treatment of OUD or pain, comprising a water-soluble film comprising electrospun fibers comprising at least one mucoadhesive polymer and LAAM or a physiologically active metabolite thereof and, optionally Mg or a salt thereof. In some aspects, the at least one mucoadhesive polymer is a copolymer comprising ethyl acrylate, methyl methacrylate and methacrylic acid ester with quaternary ammonium groups. In other aspects, the water-soluble film comprises a backing layer that is impermeable to the LAAM or a physiologically active metabolite thereof. In certain aspects, the backing layer comprises a copolymer comprising poly(ethyl acrylate, methyl methacrylate) and polyethylene glycol stearyl ether. In additional aspects, the electrospun fibers further comprise one or more of a penetration enhancer, a plasticizer, a sweetener and an antioxidant. In yet further aspects, the physiologically active metabolite is norLAAM or dinorLAMM. In additional aspects, the comprising the water-soluble film comprises Mg or a salt thereof.

In some aspects of the medicament, the levo-alpha-acetylmethadol (LAAM) or the physiologically active metabolite thereof is present in an amount of 5 mg, 10 mg, or 40 mg.

Also provided herein is an electrospun fiber comprising mucoadhesive polymers and LAAM or a physiologically active metabolite thereof and, optionally, Mg or a salt thereof.

Also provided is a method of treating opioid use disorder (OUD) or pain in a subject in need thereof, comprising administering to the subject a therapeutically effective dose of:
i) a medicament for the treatment of OUD or pain, comprising
the tablet of claim any of claims 1-8, i.e., a pellet or tablet comprising a solid core, and a solid matrix surrounding the solid core, wherein the solid matrix comprises at least one layer comprising a water-soluble polymer and levo-alpha-acetylmethadol (LAAM) or a physiologically active metabolite thereof. In some aspects, the solid core is or comprises microcrystalline cellulose. In other aspects, the water-soluble polymer is or comprises a polyethylene glycol-polyvinyl alcohol graft copolymer. In further aspects, the polyethylene glycol-polyvinyl alcohol graft copolymer and the LAAM or physiologically active metabolite thereof are present at ratio of 1:2. In additional aspects, the solid matrix comprises 2% (w/w) of LAAM or the physiologically active metabolite thereof. In some aspects, the solid matrix comprises a plurality of layers. In other aspects, the physiologically active metabolite is norLAAM or dinorLAMM. In further aspects, the solid core is or comprises Mg or a salt thereof; or
ii) a medicament for the treatment of OUD or pain, comprising
a capsule containing a plurality of pellets of or a tablet comprising a plurality of pellets comprising a solid core, and a solid matrix surrounding the solid core, wherein the solid matrix comprises at least one layer comprising a water-soluble polymer and levo-alpha-acetylmethadol (LAAM) or a physiologically active metabolite thereof. In some aspects, the solid core is or comprises microcrystalline cellulose. In other aspects, the water-soluble polymer is or comprises a polyethylene glycol-polyvinyl alcohol graft copolymer. In further aspects, the polyethylene glycol-polyvinyl alcohol graft copolymer and the LAAM or physiologically active metabolite thereof are present at ratio of 1:2. In additional aspects, the solid matrix comprises 2% (w/w) of LAAM or the physiologically active metabolite thereof. In some aspects, the solid matrix comprises a plurality of layers. In other aspects, the physiologically active metabolite is norLAAM or dinorLAMM. In further aspects, the solid core is or comprises Mg or a salt thereof; or
iii) a medicament for the treatment of OUD or pain, comprising a water-soluble film comprising electrospun fibers comprising at least one mucoadhesive polymer and LAAM or a physiologically active metabolite thereof and, optionally Mg or a salt thereof. In some aspects, the at least one mucoadhesive polymer is a copolymer comprising ethyl acrylate, methyl methacrylate and methacrylic acid ester with quaternary ammonium groups. In other aspects, the water-soluble film comprises a backing layer that is impermeable to the LAAM or a physiologically active metabolite thereof. In certain aspects, the backing layer comprises a copolymer comprising poly(ethyl acrylate, methyl methacrylate) and polyethylene glycol stearyl ether. In additional aspects, the electrospun fibers further comprise one or more of a penetration enhancer, a plasticizer, a sweetener and an antioxidant. In yet further aspects, the physiologically active metabolite is norLAAM or dinorLAMM. In additional aspects, the comprising the water-soluble film comprises Mg or a salt thereof. In some aspects of the medicament, the levo-alpha-acetylmethadol (LAAM) or the physiologically active metabolite thereof is present in an amount of 5 mg, 10 mg, or 40 mg. In further aspects, the therapeutically effective dose is from 20 to 160 mg per week. In other aspects, the therapeutically effective dose is administered 1-3 times per week. In yet further aspects, the step of administering comprises i) administering the tablet, the capsule containing a plurality of the pellets or the tablet comprising a plurality of the pellets embedded therein orally; or ii) administering the water-soluble film via buccal administration.

### BRIEF DESCRIPTION OF THE DRAWINGS

FIG. 1A-C. Schematic illustration of the preparation and application of LAAM fiber film for buccal mucosal delivery in treating Opioid Use Disorder (OUD). (A) Production of hydrophobic backing layer via solvent evaporation and deposition of LAAM fiber film using an electrospinning machine. (B) Application of LAAM fiber film to the buccal mucosa. (C) Systemic delivery of LAAM following absorption. The transmucosal delivery of LAAM via the buccal mucosa bypasses first-pass metabolism and promotes higher bioavailability, making it an effective route for OUD treatment. A protective backing layer safeguards LAAM particles from removal by mucosal secretions and saliva at the administration site.

FIGS. 2A and B. Scanning Electron Microscopy (SEM) images and drug release profiles of electrospun fiber buccal films. (A) SEM image of the optimized electrospun fiber buccal film, revealing the microstructure and morphology of the fibers. (B) The drug release profile of the electrospun fiber buccal film demonstrated the percentage of drug released over time. The release profile highlights the fast drug release characteristics of the buccal film, making it suitable for effective transmucosal drug delivery.

FIG. 3A-D. Comparative plasma pharmacokinetic profiles of LAAM and its primary metabolites, nor-LAAM and dinor-LAAM, in rabbits following different routes of administration. The plasma concentration-time curves display: (A) intravenous bolus administration of LAAM at 0.8 mg/kg (n=4); (B) oral administration of LAAM at 1 mg/kg (n=5); and buccal administration of LAAM at 4 mg using formulations (C) F2 (n=3) and (D) F5 (n=6). Data are presented as mean t standard deviation (STD), highlighting the differences in pharmacokinetic profiles for each administration route and formulation.

FIGS. 4A and B. Histology images of buccal mucosa (A) control, and (B) after 6 days of buccal film administration. Scale bar 100 μm.

FIG. 5A-C. Evaluation of buccal film properties and stability. (A) Appearance of the physical condition of the buccal film; (B) In-vitro drug release profile; and (C) Drug content analysis after long-term storage for three months under normal conditions indicates the buccal film formulation's stability and potential shelf life.

FIGS. 6A and B. (A) Drug content when increasing the ratio of polymer to drug (diphenylhydramine) and double coating by adding magnesium stearate or talc; (B) Drug content and coating time for different formulations of LAAM with Kollicoat® Protect, Soluplus®, and Kollicoat® IR.

FIG. 7. In-vitro release profile of pellets made with different coating solutions: Kollicoat® Protect, Soluplus®, and Kollicoat® IR.

FIG. 8. Production yield of pellets with coating solutions with different polymer-to-drug ratios.

FIG. 9. Drug content and coating time for different batches of pellets.

FIG. 10. Drug content of LAAM capsules with different dosage strengths.

FIG. 11. In vitro release profile of LAAM capsules.

FIGS. 12A and B. (A) Average capsule weight increases at each time point compared with month 0. (B) Average H₂O content at each time point.

FIGS. 13A and B. Drug content showed no significant change after 6 months of storage under (A) normal and (B) accelerated conditions.

FIG. 14. The LAAM capsules retained an immediate (rapid) release profile with ˜100% release in 10 minutes after 6 months of storage.

FIGS. 15 A and B. Graphical representation of solid dosage forms containing LAAM and magnesium. (A) Tablets and capsules comprising film-coated shell-core pellets; (B) core-shell tablets comprising Mg.

### DETAILED DESCRIPTION

Compositions and methods for rapidly administering LAAM, norLAAM and dinorLAMM for the treatment of opioid use disorder (OUD) and/or pain are disclosed herein. In some aspects, the compositions are based on a “core-shell” design (coated pellets or coated tablets), which is generally appropriate for oral administration. In other aspects, the compositions comprise electrospun nano/microfiber film dosage forms, which is generally appropriate for buccal administration. In each aspect, at least one of LAAM, norLAAM and dinorLAMM are present in the compositions and the compositions are designed to rapidly release the drug, for example, within about 20-60 minutes after administration. Methods of using the compositions to treat OUD and/or pain are also provided. In each aspect, Mg may optionally be included.

OUD treatment drugs, including LAAM, have been associated with the risks of the life-threatening QTc interval prolongation and potential torsade de pointes (TdP). The rapid delivery methodology described herein significantly reduces the required dosage and increases bioavailability, both of which reduce the optimal therapeutic dose that is required, and consequently adverse effects are reduced. Thus, in some aspects, Mg or a salt thereof is included in the formulations to further reduce (e.g. prevent or treat) these possible adverse effects.

In addition, the low (e.g., approximately 3 times per week) dosing regimen improves treatment by reducing the frequency of clinical visits (thus increasing patient compliance) and minimizing costs. This enables treatment providers to achieve better outcomes in patients, especially those who have not adequately responded to prior art therapies.

### Definitions

Buccal drug delivery involves the administration of the desired drug through the buccal mucosal membrane lining of the oral cavity. Buccal drug delivery specifically refers to the delivery of drugs within/through the buccal mucosa to affect local and/or systemic pharmacological actions.

Eudragit® is the brand name for a diverse range of polymethacrylate-based copolymers. It includes anionic, cationic, and neutral copolymers based on methacrylic acid and methacrylic/acrylic esters or their derivatives. These are nonionic and synthetic polyionic copolymers, including different concentrations of methacrylic acid esters, alkyl methacrylates, 2-(dimethylamino)ethyl methacrylate.

EUDRAGIT® NM 30 D is a neutral (methacrylic acid copolymer comprising poly(ethyl acrylate, methyl methacrylate) with 0.7% (PEG stearyl ether) 2:1. Eudragit® NM 30 D contains polyethylene glycol stearyl ether (0.7%).

EUDRAGIT® RL 100 is a copolymer of ethyl acrylate, methyl methacrylate and a low content of methacrylic acid ester with quaternary ammonium groups. The ammonium groups are present as salts and make the polymers permeable.

Levo-α-acetylmethadol (LAAM) [Levacetylmethadol (INN), levomethadyl acetate (USAN), OrLAAM (trade name)] is a synthetic opioid similar in structure to methadone. It has a long duration of action due to its active metabolites. LAAM acts as a p-opioid receptor agonist. It also acts as a potent, noncompetitive α3β4 neuronal nicotinic acetylcholine receptor antagonist. LAAM undergoes extensive first-pass metabolism to the active demethylated metabolite nor-LAAM, which is further demethylated to a second active metabolite, dinor-LAAM. These metabolites are more potent than the parent drug.

Kollicoat® Protect (a polyvinyl alcohol/polyethylene glycol graft copolymer) is a composition of 55-65% polyvinyl alcohol-polyethylene glycol graft copolymer, 35-45% polyvinyl alcohol and 0.1-0.3% silicon dioxide. The composition is: polyvinyl alcohol-polyethylene glycol graft copolymer 55-65%, polyvinyl alcohol 35-45%, silicon dioxide 0.1-0.3%. The CAS-number is 96734-39-3+9002-89-5+7631-86-9. Owing to the spray-drying process for Kollicoat® Protect, the polymer chains are embedded in one another to such an extent that they cannot separate. The powder has good flowability and dissolves rapidly in water, and when poured or coated onto a substrate, a film is rapidly formed by the evaporation of water.

Kollicoat® IR is a polyethylene glycol-polyvinyl alcohol graft copolymer [polyethylene glycol-polyvinyl alcohol (PEG-PVA)] that is very readily soluble in water. Kollicoat® IR is a white to faintly yellow free-flowing powder consisting of 75% polyvinyl alcohol units and 25% polyethylene glycol units. The product also contains approx. 0.3% colloidal anhydrous silica to improve its flow properties and has a molecular weight of approx. 45,000 AMU. Its CAS Number is 96734-39-3. Kollicoat® IR is readily soluble in aqueous solution and forms a clear, colorless, flexible film when cast onto a smooth surface via water evaporation. The chemical formula is shown below:

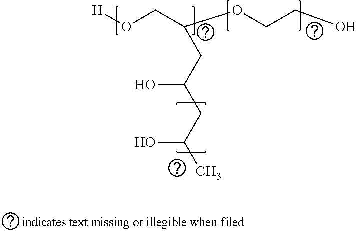

Kollidon® VA 64 (vinylpyrrolidone-vinyl acetate copolymer, also known as copovidone, copolyvidone, VP/VAc copolymer 60/40) is a copolymer of 1-vinyl-2-pyrrolidone and vinyl acetate in a ratio of 6:4 by mass. Its Cas-No is 9003-39-08.

LAAM (Levo-Alpha Acetyl Methadol, levomethadyl acetate) is the levo isomer of acetylmethadol, or α-methadyl acetate. Its chemical formula is [(3S,6S)-6-(dimethylamino)-4,4-diphenylheptan-3-yl]acetate. LAAM acts as a p-opioid receptor agonist and also as a potent, noncompetitive α3β4 neuronal nicotinic acetylcholine receptor antagonist. LAAM undergoes extensive first-pass metabolism to the active demethylated metabolite nor-LAAM (1-α-acetyl-N-normethadol) which is further demethylated to a second active metabolite, dinor-LAAM (1-α-acetyl-N,N-dinormethadol) These metabolites are more potent than the parent drug and LAAM is an alternative to methadone for the maintenance treatment of opioid dependence because is has a longer therapeutic half-life than methadone, primarily because it is metabolized to the more active metabolites, norLAAM and dinorLAAM.

Rapid release, sometimes referred to herein as “immediate release”, refers to a total drug release time from a formulation that is realized in at least about 20-60 minutes, or less.

Soluplus® is a novel solubilizer, crystallization inhibitor, and a matrix forming polymer. In particular, Soluplus® is a polymeric solubilizer with an amphiphilic chemical structure, which was particularly developed for solid solutions. The chemical structure of Soluplus® is as follows: (polyvinyl caprolactam-polyvinyl acetate-polyethylene glycol graft copolymer (PCL-PVAc-PEG)).

Talc is a naturally occurring mineral, mined from the earth, composed of magnesium, silicon, oxygen, and hydrogen. Chemically, talc is a hydrous magnesium silicate with a chemical formula of Mg₃Si₄O₁₀(OH)₂.

VIVAPUR® MCC Spheres (CAS-No. 9004-34-6) consist solely of odorless and tasteless microcrystalline cellulose (MCC) with a high degree of brightness. The MCC is derived from highly purified wood pulp and the spheres are available in a wide range of particle sizes: Grade 100, size 100-200 μm, mesh 70-140; Grade 200m size 200-350 μm, mesh 45-60; Grade 350, 355-500 μm, mesh 35-45; Grade 500, 500-710 μm, mesh 25-35; Grade 700, 710-1000 μm, mesh 18-25; Grade 1000, 1000-1400 μm, mesh 14-18. The spheres are insoluble in water and most organic solvents and have a bulk density of 800 g/l and a sphericity 0.9±0.05.

### Core-Shell Formulations: Pellets and Tablets

In one aspect, the core-shell compositions described herein comprise a particulate “pellet” or tablet dosage form comprising the drug to be delivered, e.g., LAAM, norLAAM or dinorLAAM. The pellets and tablets comprise a solid inner core surrounded or coated or double coated by an outer shell, and the drug to be delivered is typically located in the outer shell, although drug inclusion in the core is also encompassed. All components of the pellets and tablets are listed by the Food and Drug Administration (FDA) as Generally Regarded As Safe (GRAS).

The core of the pellet or tablet generally comprises a carrier material which plays the role of filler and/or solid diluent. In some aspects, the core is or includes a physiologically inert material and serves as a carrier. In other aspects, the core is or includes a physiologically active agent such as Mg or a salt thereof and serves as both a carrier and a medicament. Examples of materials which form or are included in the core include but are not limited to: microcrystalline cellulose, calcium phosphate, lactose, starch, various forms of Mg and salts thereof discussed elsewhere herein, and derivatives thereof. The core material can be formed using methods that are known in the art or may be readily purchased commercially. In one aspect, the core material is microcrystalline cellulose. In another aspect, the core is or comprises Mg or a salt thereof.

The drug-polymer solution generally comprises at least one polymer, examples of which include but are not limited to: Soluplus® (polyvinyl caprolactam-polyvinyl acetate-polyethylene glycol graft copolymer), Kollicoat® Protect (a polyvinyl alcohol/polyethylene glycol graft copolymer which is a composition of 55-65% polyvinyl alcohol-polyethylene glycol graft copolymer, 35-45% polyvinyl alcohol and 0.1-0.3% silicon dioxide), Kollicoat® IR (a polyethylene glycol-polyvinyl alcohol graft copolymer “polyethylene glycol-polyvinyl alcohol (PEG-PVA)” consisting of 75% polyvinyl alcohol units, 25% polyethylene glycol units and approx. 0.3% colloidal anhydrous silica), Kollidon® VA 64 (vinylpyrrolidone-vinyl acetate copolymer, a copolymer of 1-vinyl-2-pyrrolidone and vinyl acetate in a ratio of 6:4 by mass), hydroxypropryl methylcellulose, hydroxypropryl cellulose, cellulose acetate phthalate, hydroxyethyl cellulose, shellac, polyvinyl acetate, ethyl cellulose, polyvinylpyrrolidone, and derived copolymers. In some aspects, a polyethylene glycol-polyvinyl alcohol graft copolymer is used.

The ratio of polymer to drug (e.g., LAAM, norLAAM or dinorLAAM) is generally in the range of about 10:0.1, such as about 10, 9, 8, 7, 6, 5, 4, 3, 2 or 1 to about 0.1, 0.2, 0.3, 0.4, 0.5, 0.6, 0.7, 0.8, 0.9 or 1.0. In some aspects, the ratio of polymer to drug is 2:1. Ratios are selected so as to permit a practical amount of drug to be included in the coating and to yield a hard, durable yet water-soluble, rapid or immediate release coating on the pellet core.

Generally, the amount of drug that is present in e.g., 1 gram of pellets or tablets ranges from about 5 to about 200 mg, such as about 5, 10, 15, 20, 25, 30, 35, 40, 45, 50, 55, 60, 65, 70, 75, 80, 85, 90, 95, 100, 105, 110, 115, 120, 125, 130, 135, 140, m 145, 150, 155, 160, 165, 170, 175, 180, 185, 190, 195 or 200 mg/g. In some aspects, about 13.1, 26.2 or 104.8 mg of LAAM, norLAAM or dinorLAAM are present in one gram of pellets.

The pellets and tablets comprising a drug are typically made by coating the core particle with 1-2 or more layers of the drug-polymer solution. Methods of coating are known in the art, and include, for example, fluid bed coating, compression coating etc. Preferably, the method of coating is fluid bed coating.

It is noted that if more than one layer of drug-polymer solution is desired, in some aspects, talc, magnesium stearate, or other solid ingredients with glidant properties are added to make the particles less “sticky”, or less adhesive, and thus avoiding aggregation during the coating, storage, transportation, handling, and other unit operation processes. Due to the hydrophilic nature of polymers used for coating, a hydrated layer of polymer on the surface of the shell may lead to “stickiness” even when the product is fully dried (e.g., air humidity). Glidant ingredients will generally be positioned on the interface of the polymeric shell and air, thus reducing moisture uptake and “stickiness”.

Drying (dehydration) of the polymer plus drug solution after coating forms a hard but readily water, and hence physiologically, soluble polymer matrix coating (shell) on the outside of the core particles.

The pellets are substantially spheroidal in shape although the shapes can be irregular. The dimensions of the pellets are generally in the range of from about 0.3 mm to about 3.0 mm and are more specifically from about 0.6 mm to about 2.0 mm such as about 0.8-1.2 mm or about 1.0 mm.

The coated tablets can be of any convenient size and shape that is suitable for oral administration, e.g., so that a subject can readily swallow a suitable dose. Examples include but are not limited to: round, ovoid, caplet form, etc.

For delivery to a subject, the pellets are typically loaded into a capsule or embedded in a tablet, although other carrier/delivery means are not excluded.

Types of capsules which may be used include any that are known to be safe for administration and which dissolve rapidly upon contact with physiological fluids. Examples include but are not limited to: those of animal origin such as gelatin from collagen, and plant-derived polysaccharides or their derivatives such as carrageenans and modified forms of starch and cellulose. Other ingredients can be added including plasticizers such as glycerin or sorbitol to decrease the capsule's hardness, coloring agents, opacifiers, preservatives, disintegrants, lubricants and surface treatments.

In other aspects, the pellets are embedded in a “tablet”, which can take any form e.g., spherical, ovoid, “caplets”, etc. The pellets are embedded within the tablet and some pellets may also be exposed at the surface of the tablet. These tablets are typically prepared by compression of a mixture of pre-made pellets and tableting ingredients in the powder form. Typically, most of the tablet composition, for both tablets with embedded pellets and tablets coated with a drug-polymer matrix, is made of a filler material such as microcrystalline cellulose and its derivatives, lactose, etc. Tablets may also contain a variety of other ingredients in smaller quantities that play functions of binders, disintegrants, lubricants, glidants, edulcorates, etc. In some aspects, the tablets comprise or are comprised of Mg or a salt thereof.

The dose of LAAM or breakdown product thereof in a single capsule or tablet is generally in the range of about 5 to about 150 mg, such as about 5, 10, 15, 20, 25, 30, 35, 40, 45, 50, 55, 60, 65, 70, 75, 80, 85, 90, 95, 100, 105, 110, 115, 120, 125, 130, 135, 140, 145 or 150 mg. In some aspects, the dose is from about 40 to 140. Preferably, the LAAM dose varies between 20-160 mg a week (given e.g., 1, 2 or 3 times) depending on the patient's need and the severity of the OUD. Thus, about 20, 40, 60, 80, 100, 120, 140 or 160 mg is administered per week.

When norLAAM or dinorLAAM are administered, the doses and the dosing frequency are similar to that of LAAM but the dose may be lower (e.g. on the lower end of the range for LAAM or even less) and the frequency may be lower (e.g. also on the lower end of the range for LAAM or less). Those of skill in the art will recognize that the precise dosages and frequencies vary depending on e.g., the weight, gender, age, overall health, etc. of the patient and are determined e.g., during clinical trials, and finalized and prescribed under the supervision of a medical professional.

Once ingested by a subject, the capsule shell is readily dissolved and the pellets are released or tablet readily disintegrates and the coating is rapidly dissolved, releasing the drug. At this stage, the drug is available for absorption and becomes bioavailable. The formulations described herein, whether in capsule or tablet form, are rapid release formulations, i.e., the drug is entirely (completely, fully) released into the circulatory system of a subject to whom a formulation has been administered within at least about 1-60 minutes, such as about 1, 5, 10, 15, 20, 25, 30, 35, 40, 45, 50, 55, or 60 minutes. Preferably, the drug is released in at least about 5 to 30 minutes, such as about 5, 10, 15, 20, 25 or 30 minutes. More preferably, the drug is completely released within from about 5-20 minutes.

### Film Formulations

Film or sheet formulations for transmucosal delivery of LAAM, norLAAM or dinorLAAM are also provided. The films are a novel formulation of electrospun fibers comprising LAAM. The electrospun fibers have mucoadhesive properties suitable to effect the transport of LAAM, norLAAM or dinorLAAM across the buccal mucosa.

The films are comprised of mucoadhesive polymers that are both rate-controlling and rapid release. Examples of suitable rate controlling polymers include but are not limited to: water-soluble hypromellose (HPMC), polyvinylpyrrolidone (PVP), water-insoluble ethyl cellulose (EC), polyethylene glycol (PEG), Eudragit® RL and propylene glycol (PG), etc. Since polymers like HPMC, EC, PEG, Eudragit® RL and PG cannot form fibers alone, PVP is typically used in formulations in an amount ranging from about 23 to about 45% (w/w) of total formulation weight, such as from about 20, 21, 22, 23, 24, 25, 26, 27, 28, 29, 30, 31, 32, 33, 34, 35, 36, 37, 38, 39, 40, 41, 42, 43, 44 or 45%. Generally, at least two (for example, 2, 3, 4, 5, or 6) of these polymers are combined to create drug-loaded nanofiber formulations, examples of which include but are not limited to: PVP-HPMC-PG, PVP-EC-PG, PVP-HPMC-EC-PG, PVP-RL-PG, PVP-PEG-RL-PG, etc.

The ratio of each polymer that is present in a nanofiber formulation generally ranges from about 1 to about 100%, such as about 1, 5, 10, 15, 20, 25, 30, 35, 40, 45, 50, 55, 60, 65, 70, 75, 80, 85, 90, 95 or 100%, including all single digits in between (e.g. 1, 2, 3, 4, 5 . . . 96, 97, 98, 99, 100) and decimal fractions thereof in between to the nearest 0.1%, (e.g. 1.1, 1.2, 1.3, 1.4, 1.5 . . . 99.5, 99.6, 99.7, 99.8, 99.9 and 100.0%). “100%” or “100.0%” indicates that only one type of polymer is used in a preparation.

Individual films may be any shape but are generally substantially circular, oval, rectangular, square, etc. The sizes of individual films may vary but generally the area ranges from about 4.2 to about 4.25 cm². The films typically have a thickness of from about 0.8 to about 0.9 mm.

The amount of LAAM, norLAAM or dinorLAAM in a single dose of film is generally in the range of about 1 to about 50 mg, such as about 1, 5, 10, 15, 20, 25, 30, 35, 40, 45 or 50 mg. In some aspects, the dose is from about 20 to 40 mg on a single film that is administered. It is noted that more than one film may be administered to a subject at a time, if higher total doses are required.

In some aspects, the films further comprise one or more excipients. The one or more excipients may be present within (as a component of) the electrospun fibers themselves or they may be present outside the electrospun fibers as a component of the aggregated fibers which form the film. Exemplary excipients include but are not limited to: penetration enhancers, plasticizers, sweeteners, antioxidants, various flavorings, colorants, mucoadhesion enhancers, saliva stimulating agents, and preservatives, etc.

Exemplary mucoadhesion enhancers may be added. Mucoadhesion enhancers are generally positively charged nanoparticles which increase the interaction between mucin (in the mucous membrane) and a film, thereby increasing drug bioavailability. Exemplary mucoadhesion enhancers include but are not limited to: Eudragit® RL (C₁₁H₂₁NO₄, N,N-dimethylmethanamine; 2-methylprop-2-enoic acid); chitosan and derivatives thereof such as chitosan-4-thiobuthylamidine; Carbopol 980 (CAS number 139637-85-7) is a crosslinked polyacrylic acid polymer; Carbopol 974P (CAS Number: 9003-01-04; Formula: C₃H₄O); polycarbophil (CAS Number: 9003-97-8), cross-linked polyacrylic acid with divinyl glycol and a calcium counter-ion; ethyl cellulose (CAS 9004-57-3) Exemplary penetration enhancers include but are not limited to: hydrophobin polypeptides, sodium dodecyl sulfate, ammonium lauryl sulfate, sodium lauryl sulfate, cetyl trimethyl ammonium bromide, cetyl pyridinium chloride, benzethonium chloride, cocamidopropyl betaine, cetyl alcohol, oleyl alcohol, octyl glucoside, decyl maltoside, sodium octyl sulfate, sodium decyl sulfate, sodium tetradecyl sulfate, sodium heptadecyl sulfate, sodium eicosyl sulfate, nicotine sulfate, sodium taurocholate sulfate, dimethyl sulfoxide, sodium tridecyl phosphate, decyl dimethyl ammonium propane sulfonate, oleyl chembetine, myristyl dimethyl ammonium propane sulfonate, chlorinated benzylpyridines, dodecyl pyridine chloride, cetyl pyridinium chloride, benzyl dimethyldodecylammonium chloride, benzyl dimethylmyristylammonium chloride, benzyl dimethylstearoylammonium chloride, octyl trimethylammonium bromide, dodecyltrimethylammonium bromide, polysorbate 20, polysorbate 40, polysorbate 60, or polysorbate 80, limonene, cymene, pinene, camphor, menthol, complene, phellandrene, sabinene, terpinene, borneol, eucalyptol, geraniol, linalool, piperitone, terpineol, eugenol acetate, safrole, benzyl benzoate, lupinene, beta-caryophyllene, eucalyptol, hexanoic acid, octanoic acid, decanoic acid, undecanoic acid, dodecanoic acid, tridecanoic acid, myristic acid, palmitic acid, stearic acid, oleic acid, linoleic acid, linolenic acid, cholic acid, ethyl undecanoate, methyl laurate, methyl myristate, isopropyl palmitate, palmityl palmitate, diethyl sebacate, glycerol monolaurate, glycerol monooleate, ethylpiperazine carboxylate, sodium lauroyl sarcosinate, sorbitan monooleate, octoxynol-9, diethyl sebacate, sodium polyacrylate with molecular weight of 2500000MW, octyldodecanol, pyrrolidone, bupivacaine, tetracaine, procaine, proparacaine, propoxycaine, dicaine, cyclomecaine, chloroprocaine, benzocaine, lidocaine, prilocaine, levobupivacaine, ropivacaine, dibucaine, articaine, ticarcine, etidocaine, mepivacaine, piperocaine, and tricaine, and polyethylene glycol, etc.

Examples of suitable plasticizers include but are not limited to: dipropylene glycol; sorbitol; glycerin; various conventional plasticizers such as phthalates, hydrogenated phthalates, aliphatic esters of dicarboxylic acids, polymeric esters of dicarboxylic acids, citrates, sucrose esters, ketal ester esters, phosphates, alkyl phenol sulphonates, pyrrolidones, and the like; biologically based plasticizers derived from fatty acids containing epoxy functional groups; etc.

Exemplary sweeteners, which may also be used in the pellet and tablet formulations discussed herein, include but are not limited to: various natural sweeteners found in nature which may be in raw, extracted, purified, or any other form, singularly or in combination thereof including but not limited to: sucrose, fructose, glucose, syrups or sugars extracted or made from plants or fruits (e.g., agave syrup, maple syrup or sugar, etc.), and the like. Other natural sweeteners that have a sweetness potency greater than that of sucrose and yet have less calories include but are not limited to: rebaudioside A, rebaudioside B, rebaudioside C, rebaudioside D, rebaudioside E, rebaudioside F, rubusoside M, dulcoside A, dulcoside B, rubusoside, stevia, stevioside, mogroside IV, mogroside V, Luo Han Guo sweetener, siamenoside, monatin and its salts (monatin SS, RR, RS, SR), curculin, glycyrrhizic acid and its salts, thaumatin, monellin, mabinlin, brazzein, hemandulcin, phyllodulcin, glycyphyllin, phloridzin, trilobatin, baiyunoside, osladin, polypodoside A, pterocaryoside A, pterocaryoside B, mukurozioside, phlomisoside I, periandrin I, abrusoside A, and cyclocarioside I. Non-limiting examples of synthetic sweeteners suitable for use include but are not limited to: sucralose, potassium acesulfame, aspartame, alitame, saccharin, neohesperidin dihydrochalcone, cyclamate, neotame, N—[N-[3-(3-hydroxy-4-methoxyphenyl)propyl]-L-a-aspartyl]-L-phenylalanine 1-methyl ester, N—[N-[3-(3-hydroxy-4-methoxyphenyl)-3-methylbutyl]-L-aspartyl]-L-phenylalanine 1-methyl ester, N—[N-[3-(3-methoxy-4-hydroxyphenyl)propyl]-L-aspartyl]-L-phenylalanine 1-methyl ester, salts thereof, etc.

In some aspects, antioxidants are not included. In other aspects, they are included. Examples of suitable antioxidants, which may also be used in the pellet and tablet formulations discussed herein, include but are not limited to: alkylated monophenols, 2,6-di-tert-butyl-4-methylphenol, alkylthiomethylphenols, 2,4-dioctylthiomethyl-6-tert-butylphenol, alkylated hydroquinones, 2,6-di-tert-butyl-4-methoxyphenol, hydroxylated thiodiphenyl ethers, for example, 2,2′-thiobis (6-tert-butyl-4-methylphenol), alkylidenebisphenols, 2,2′-methylene-bis (6-tert-butyl-4-methylphenol), benzyl compounds, 3,5,3′,5′-tetraterc-butyl-4,4′-dihydroxydibenzyl ether, hydroxybenzylated malonates, 2,2-bis(3,5-di-tert-butyl-2-hydroxybenzyl) dioctadecyl malonate, hydroxybenzyl aromatics, 1,3,5-tris (3,5-di-tert-5) butyl-4-hydroxybenzyl)-2,4,6-trimethylbenzene, triazine compounds, 2,4-bisoctylmercapto-6-(3,5-di-tert-butyl-4-hydroxyanilino)-1,3,5-triazine, phosphonates and phosphonites, dimethyl 2,5-di-tert-butyl-4-hydroxybenzyl phosphonate, acylaminophenols, 4-hydroxylauranylide, esters of the acid p-(3,5-diterc-butyl-4-hydroxyphenyl) propionic, pentaerythritol tetrakis (3-(3,5-di-tert-butyl-4-hydroxyphenyl) propionate), 3-(3,5-di-tert-butyl-4-hydroxyphenyl) propionate octadecyl, p-(5-tert-butyl-4-hydroxy-3-methylphenyl) propionic acid, p-(3,5-dicyclohexyl-4-hydroxyphenyl) propionic acid esters 3,5-diterc-butyl-4-hydroxyphenylacetic acid with mono- or polyhydric alcohols, p-(3,5-diterc-butyl acid amides)-4-hydroxyphenyl) propionic, N,N′-bis (3,5-diterc-butyl-4-hydroxyphenyl-propionyl) hexamethylene diamine, vitamin E (tocopherol), vitamin C, and derivatives thereof. Mixtures of antioxidants can also be used. The amount antioxidant used are between about 0.01 and about 10 parts by weight, advantageously between 0.1 and 5 parts by weight, and in particular between 0.1 and 3 parts by weight, based on 100 parts by total weight of the film.

Exemplary flavorings that may be used, and which may also be used in the pellet and tablet formulations discussed herein, include but are not limited to: various synthetic and natural fruit and berry flavorings; various plant-derived flavorings (cinnamon, vanilla, cacao, coffee, mint, etc.); liquor-based flavorings such as rum, whiskey, etc.; nut-based flavorings (e.g. almond, walnut, peanut, etc.); and the like. Mixtures of these may also be employed.

Exemplary colorants that may be added, and which may also be used in the pellet and tablet formulations discussed herein, include but are not limited to: natural colorant, a combination of natural colorants, an artificial colorant, a combination of artificial colorants, or a combination of natural and artificial colorants. Suitable examples of natural colorants approved for use in food include annatto (reddish-orange), anthocyanins (red to blue, depends upon pH), beet juice, beta-carotene (orange), beta-APO 8 carotenal (orange), black currant, burnt sugar; canthaxanthin (pink-red), caramel, carmine/carminic acid (bright red), cochineal extract (red), curcumin (yellow-orange); lac (scarlet red), lutein (red-orange); lycopene (orange-red), mixed carotenoids (orange), monascus (red-purple, from fermented red rice), paprika, red cabbage juice, riboflavin (yellow), saffron, titanium dioxide (white), and turmeric (yellow-orange). Suitable examples of artificial colorants approved for food use in the United States include FD&C Red No. 3 (Erythrosine), FD&C Red No. 40 (Allure Red), FD&C Yellow No. 5 (Tartrazine), FD&C Yellow No. 6 (Sunset Yellow FCF), FD&C Blue No. 1 (Brilliant Blue), FD&C Blue No. 2 (Indigotine). Artificial colorants that may be used in other countries include Cl Food Red 3 (Carmoisine), Cl Food Red 7 (Ponceau 4R), Cl Food Red 9 (Amaranth), Cl Food Yellow 13 (Quinoline Yellow), and Cl Food Blue 5 (Patent Blue V). Food colorants may be dyes or pigments which are powders, granules, or liquids that are soluble in water.

### Film Characteristics

Size and Morphology of the fibers: The fibers generally have a diameter ranging from about 0.10 to about 0.50 μm, e.g., about 0.10, 0.15, 0.20, 0.25, 0.30, 0.35, 0.40, 0.45, or 0.50 μm, including all decimal fractions in between to 0.001 decimal places.

Mucoadhesive strength: The mucoadhesive strength of a film of the present disclosure generally ranges from about 0.05 to about 2.0 Newtons (kg·m/s²), such as about 0.05, 0.10, 0.20, 0.30, 0.40, 0.50, 0.6, 0.7, 0.8, 0.9, 1.0, 1.1, 1.2, 1.3, 1.4, 1.5, 1.6, 1.7, 1.8, 1.9 or 2.0 Newtons. In some aspects, the mucoadhesive strength ranges from about 0.05 to 1.1 Newtons.

### Backing Layer

In some aspects, the films also comprise a backing layer that is impermeable to the drug in the film. The purpose of the backing layer is to prevent or decrease release of the drug on the side of the film that faces away from the mucosa, i.e., to prevent release into the gastrointestinal tract. This fosters or promotes unidirectional release of the drug into the buccal mucosa and delivery locally and into the blood stream.

Several materials may be used to form the backing material, including but not limited to ethyl cellulose, Eudragit® NM 30 D (e.g., a dispersion which contains 30% of a dry neutral copolymer based on ethyl acrylate and methyl methacrylate and 0.7% macrogol stearyl ether as an emulsifier, this aqueous dispersion is miscible with water at any ratio, retaining the milky-white appearance); etc.

### Methods of Making the Films

Methods of making the polymeric films disclosed herein are also provided. The methods generally comprise a step of preparing a liquid polymer solution of the one or more, usually two or more, polymers, in suitable solvents. Suitable solvents include, for example, water, alcohol (e.g., methanol, ethanol, etc.), and various mixtures thereof. The exact type of solvent(s) used depends on the solubility of the polymers, as will be readily understood by those of skill in the art. Frequently, mixtures of ethanol and water are used. Polymer solutions are prepared and mixed with LAAM, norLAAM or dinorLAAM, and then drug-polymer solutions are stirred to mix thoroughly, e.g., for from about 1-5 hours or longer before electrospinning.

Exemplary polymer solutions include: polyvinyl pyrrolidone (PVP) and ethyl cellulose (EC) dissolved in ethanol at 15% (w/w) and 5% (w/w) respectively; 2% (w/w) hypromellose (HPMC) dissolved in 50% ethanol-water (v/v) solution; Eudragit® RL 10% (w/w) dissolved in ethanol, PEG 15% (w/w) dissolved in water, etc.

The preparation of LAAM, norLAAM or dinorLAAM electrospun fiber films involve loading a liquid polymer-drug solution into a suitable vessel, such as a syringe with a needle for electrospinning. For example, a 10 mL syringe with a 22G needle can be used. The electrospinning process is generally conducted with the following parameters:
5-20 kV, such as about 5, 10, 15 or 20 kV, typically 15 kV;
Flowrate of 500 to 3000 μL/h, such as about 500, 1000, 1500, 2000, 2500 or 3000 μL/h, typically 1000-2000 μL/h;
Temperature range from about 15-50° C., such as about 15, 20, 25, 30, 35, 40, 45 or 50° C., and typically from about 25-30° C.;
Humidity range from about 10 to 40%, such as about 10, 15, 20, 25, 30 35 or 40%, and typically from about 20-30%.

If a backing layer is included, the backing layer is formed by casting a layer of a solution of the backing material onto a flat, non-porous surface at a depth of from about 0.1 to about 0.5 mm, such as about 10.1, 0.2, 0.3, 0.4 or 0.5 mm. Typically, the depth is from about 0.2 to about 0.4 mm, e.g., about 0.2, 0.3 or 0.4 mm. The solution is allowed to dry (dehydrate) and is then attached to the film, e.g., by electrospinning the film directly onto the backing layer. In other words, after the backing film is formed, it is placed on the surface of the electrospinning machine, and the electrospun fibers are formed on the exposed top surface of the backing layer.

Once administered to a subject, the film is readily dissolved, releasing the drug. The film formulations are rapid release formulations, i.e., the drug is entirely (completely, fully) released into the circulatory system of a subject who applies the film within at least about 1-60 minutes, such as about 1, 5, 10, 15, 20, 25, 30, 35, 40, 45, 50, 55, or 60 minutes. Preferably, the drug is released in at least about 5 to 20 minutes, such as about 5, 10, 15, or 20 minutes.

### Optional Addition of Magnesium (Mg)

Embodiments of the disclosure provide solid dosage forms to deliver LAAM, norLAAM or dinorLAAM orally while displaying similar or improved product performance when compared with a LAAM, norLAAM or dinorLAAM solution product previously available on the market. Embodiments provide the use of polymer-based thin coatings which enable the preparation of dried amorphous LAAM, norLAAM or dinorLAAM dispersions with instantaneous release capabilities, while simultaneously delivering Mg (e.g., Mg and/or at least one salt thereof) to reduce concerns of QTc interval prolongation and electrospum dosage forms for buccal administration. The dosage forms include:
i) Capsules filled with core-shell coated pellets, in which LAAM, norLAAM or dinorLAAM is retained in the amorphous coating layer for instantaneous release and the core contains Mg; The doses of LAAM, norLAAM or dinorLAAM and Mg can be easily adjusted to the clinician's need by adding more or fewer pellets into the capsule. This dosage form enables industrial-scale production as well as compounding in pharmacies for precise dose adjustment.
ii) Tablets containing core-shell coated pellets embedded therein, in which LAAM, norLAAM or dinorLAAM is retained in the amorphous coating layer for instantaneous release and the core contains Mg (e.g., one or more salts thereof). The shell coating may also contain Mg (e.g., one or more salts thereof) to provide extra dosing of this component based on the clinician's need. This dosage form provides less dose flexibility as the tablet is manufactured to a final desired dose, but it is also advantageously less subject to tampering.
iii) Core-shell tablets, in which LAAM, norLAAM or dinorLAAM is retained in an amorphous coating on the outside of the tablet for instantaneous release and the tablet core contains Mg (e.g., one or more salts thereof). The shell coating may also contain Mg to provide extra dosing of this component based on the clinician's need. This dosage form provides less dose flexibility, but it is easier and cheaper to manufacture on a large scale and is also advantageously less subject to tampering.
iv) Films for buccal administration, the films comprising or comprised of electrospun nano/microfiber films which comprise LAAM, norLAAM or dinorLAAM and optionally, Mg. In some aspects, the Mg (e.g., one or more salts thereof) is included in the composition that is electrospun. In other aspects, the Mg or one of more salts thereof is attached to the electrospun fibers as a coating In yet other aspects, the films comprise a backing (described elsewhere herein) and the backing contains Mg embedded therein or attached thereto on the side of the backing that attaches to the electrospun film. In other words, a layer comprising Mg is located (sandwiched) in between the backing and the electrospun film.

In some aspects, the magnesium is immediately released from the dosage forms disclosed herein. In other aspects, Mg release is sustained, in contrast to the immediate release of the drug. Thus, immediate and sustained Mg release pellets and tablets are made with magnesium as the core, and a drug-carrier solid dispersion as the shell. In addition, tablets in which drug coated pellets are embedded may comprise Mg in the core of the embedded pellets, in the filler material that surrounds the embedded pellets, or both. Uncoated magnesium pellet cores, tablet cores and/or tablet fillers comprising various amounts of Mg can be prepared for dose adjustment purposes. The amount of Mg in a core or filler material generally ranges from about 1-100%, such as about 1, 5, 10, 15, 20, 25, 30, 35, 40, 45, 50, 55, 60, 65, 70, 75, 80, 85, 90, 95 or 100%.

The dose of Mg that is administered generally ranges from about 100 to 100 mg per dose, depending on the gender, weight, age and overall health of the subject, as well as the dosage of drug that is administered. For example, a dose of from about 100, 150, 200, 250, 300, 350, 400, 450, 500, 550, 600, 650, 700, 750, 800, 850 900, 950 or 1000 mg may be administered. In some aspects, the dose is adjusted to maintain a normal or near normal range for blood magnesium level of from about 1.7 to 2.2 mg/dL (0.85 to 1.10 mmol/L).

Magnesium immediate and sustained release pellets and tablets are designed using a polymer matrix system with different amounts of a polymer matrix comprising the drug and bulking agents and stabilizers including hydroxypropylmethylcellulose, microcrystalline cellulose, polyvinylpolypyrrolidone, polyethylene glycol, stearic acid, calcium stearate, etc., in the core or filler, as described elsewhere herein. For the LAAM, norLAAM or dinorLAAM coating, the polymer matrix formulations also as described elsewhere herein are used. Magnesium pellets are generally manufactured by wet granulation. In some aspects, the optimal magnesium immediate and sustained release pellets are coated with a frug-carrier solution using a VFC-LAB Micro Fluid Bed Coater and a bottom-spray (Wurster) coating method.

Exemplary forms of Mg that are used to prepare the formulations disclosed herein include but are not limited to: magnesium oxide, citrate, chloride, sulfate, glycerophosphate, gluconate, lactate, stearate, acetate and the like, and the like. In some aspects, more than one Mg salt is included in a single type of formulation. For example, one type of salt may be more suitable for inclusion in a coating while another type of Mg salt may be more suitable for inclusion in the core of a formulation.

Methods of Treating and/or Preventing OUD and/or Pain

Also provided herein are methods of preventing and/or treating and/or alleviating one or both of at least one symptom of OUD and/or at least one symptom of pain.

For an individual addicted to opioids, symptoms of withdrawal are debilitating and very difficult to cope with, leading to frequent relapses and the reuse of opioids. Signs and symptoms of opioid withdrawal include drug craving, anxiety, restlessness, gastrointestinal distress, diaphoresis, and tachycardia. Such symptoms are induced by both spontaneous opioid withdrawal (a patient who is physiologically dependent on opioids reduces or stops opioid use abruptly) and precipitated opioid withdrawal (a patient who is physiologically dependent upon opioids and who has or recently had opioids in their system is administered an opioid antagonist (e.g., naloxone, naltrexone, or nalmefene) or an opioid partial agonist (e.g., buprenorphine).

In various aspects, patients treated with the drug dosage forms described herein present for opioid withdrawal management and include those with untreated opioid use disorders, those on methadone or buprenorphine maintenance who are ending this treatment voluntarily or not, those currently using opioids who are to be initiated on extended-release drug therapy, and those ending chronic opioid treatment for pain management. The use of the present dosage forms under supervised opioid withdrawal reduces the severity of withdrawal symptoms. In some aspects, the present drug dosage forms replace e.g., methadone or buprenorphine maintenance therapy.

The tablets and capsules disclosed herein are generally administered orally, i.e., by swallowing. The films described herein are designed for delivery across mucocutaneous linings, especially via buccal delivery through the oral mucosa such as under the tongue, on the tongue, against the interior of the cheek, etc.

The recipient of the drug dosage form(s) disclosed therein is usually a mammal, and may be a human, although veterinary applications are also encompassed, e.g., to treat pain or to act as a sedative in animals. The amount that is administered varies based on several factors, as will be understood by those of skill in the art. For example, the dose and frequency of administration varies according to the type of condition or disease for which the opioid is administered, the severity of the opioid side effects, the opioid that is administered, gender, age, weight, general physical condition, genetic background, etc. of the individual, as well as whether or not the individual has other diseases or conditions that might impinge on the treatment. Generally, the dose will be in the range of from about 0.01 to about 1000 mg/kg of body weight (e.g., about 0.1, 0.5, 1.0, 10, 20, 30, 40, 50, 60, 70, 80, 90, 100, 125, 150, 175, 200, 225, 250, 275, 300, 325, 350, 375, 400, 425, 450, 475 or 500 mg/kg, etc.).

Generally the desired dose of e.g., LAAM, norLAAM or dinorLAAM is from about 25 to about 150 mg per dose, such as about 25, 30, 35, 40, 45, 50, 55, 60, 65, 70, 75, 80, 85, 90, 95, 100, 125, 130, 135, 140, 145 or 150 mg per dose, with the amount typically being in the range of from about 40 to about 140 mg per dose, such as about 40, 45, 50, 55, 60, 65, 70, 75, 80, 85, 90, 95, 100, 105, 110, 115, 120, 125, 130, 135, or 140 mg per dose.

Further, especially for the prevention and/or treatment of opioid addiction, the dosage may vary with time. For example, initial doses may be relatively high (e.g., near or at 140) so as to eliminate or greatly reduce at least one symptom of withdrawal. Over time, for example, over a few weeks or months, the dose is decreased from 140 to e.g., 120, then 100, etc. until the low dose of 40 is reached, as long as the withdrawal symptoms are manageable, and the patient is compliant. Thereafter, the dose may be further lowered, or discontinued, and/or the time between doses may be extended, etc., according to protocols that are well known to those of skill in the art.

For the prevention and/or treatment of pain, the dosage may be determined based on the condition that has caused the pain. For example, if the subject is a terminally ill patient (e.g., with cancer or another painful condition that is not curable), the dosage may be relatively high (e.g., starting at about 100 mg per dose and increasing as needed for comfort) and may increase with time as the disease progresses. The dose may even be determined by the user. Alternatively, for short term pain, such as for pain after an operation that will dissipate naturally as healing progresses, the initial dose may be high (e.g. about 140 mg per dose) or relatively moderate (e.g., less than 140 mg, such as about 50-100 mg) and may be tapered off (decreased) to about 40 mg as healing occurs, or a single dose level may be prescribed for the short term, e.g., for 3-5 days postop, or for 1, 2, 3, or 4 weeks, and/or simply on and “as needed” based on the patient's own judgement. Types of pain that can be prevented or treated include but are not limited to: acute pain such as postsurgical pain, pain resulting from an accident or accidental injury, and the like; and chronic pain such as pain resulting from chronic, incurable conditions such as uncurable cancers (e.g., multiple myeloma, and bone metastases from solid tumors), uncurable bone diseases, arthritis, and the like. Other conditions that elicit pain that may be prevented, treated or alleviated include but are not limited to inflammatory pain, muscle spasms, migraine headaches, cancer-related pain, chronic pelvic pain, complex regional pain syndrome, costochondritis, spine-related pain, etc.

The dosage formulations described herein may be administered alone, as a single active agent, or in combination with one or more other active agents. “In combination” refers to both sequential administration (e.g., one agent is administered after the other), and administration of a mixture of both active agents. Exemplary combinations include but are not limited to combinations of a drug dosage form as disclosed therein with morphine, oxycodone, methadone, etc. In some aspects, the combinations are fixed dose combinations. In addition, the dosage forms may be administered in conjunction with other treatment modalities such as other medicaments (e.g., other pain medications, other agents that counteract unwanted opioid-induced side effects), chemotherapeutic agents, other types of therapy (e.g., exercise, surgery, psychotherapy, etc.), radiation therapy, surgery, and the like.

Those of skill in the art will recognize that, to be beneficial, “prevention”, “preventing”, “treatment” and “treating”, need not completely eliminate all unwanted symptoms of, e.g., pain, OUD, etc. Much benefit can accrue from the lessening of symptoms to a more manageable or tolerable level, or to a level at which other agents can be used to provide further relief.

In addition, those of skill in the art will recognize that in some aspects, “prevention” refers to keeping at least one symptom of OUD or pain from happening or arising (e.g., administering the medicaments described herein during or directly after surgery and before the effects of a local anesthetic subside, administering the medicaments described herein to a subject who intends to withdraw from opioid use but has not yet experienced a withdrawal symptom, etc.). In some aspects, “treating” refers to administering the medicaments described herein after at least one symptom of OUD or pain has begun in order to lessen, decrease or eliminate (e.g., alleviate) the at least one symptom.

### Methods of Making the Films, Pellets, Tablets and Capsules

Methods of making the films and pellets disclosed herein are also provided, as are methods of making the tablets and capsules that contain the pellets.

Briefly, the films are typically made by electrospinning a solution comprising at least one suitable water soluble, mucoadhesive polymer and LAAM (or a physiologically active catabolic breakdown product thereof) to form a substantially flat sheet or mat of polymer-drug fiber film. In some aspects, a backing is attached to one side of the film. The method of adhering the backing layer does not need to be a separate process, as the fibers naturally dry and attach themselves to the surface of the backing layer during the process of electrospinning. It's noteworthy that this adhesion is augmented by the similar chemical structures of both the fiber and backing layer. For example, in some aspects, the fiber film includes Eudragit® RL, while the backing layer is comprised of Eudragit® NM 30 D. This chemical compatibility facilitates a successful integration between the fiber film and the backing layer. However, in other aspects, it may be beneficial to apply force to the product to further enhance the attachment.

The resulting film plus backing is then cut to a size that is suitable for administration (described elsewhere herein) and individually packaged, e.g., in a blister pack or other container that is appropriate for storage and provision to a user. Much of the cutting and packaging is automated but some steps may be done by hand as needed. Dosage forms of packaged films are also encompassed herein.

The pellets disclosed herein are generally manufactured by coating an inert core particle with a solution comprising a suitable water-soluble polymer and LAAM (or a physiologically active catabolic breakdown product thereof) using e.g., fluid bed or other technology known to those of skill in the art, thereby forming a polymer matrix on the outside of the core particle. The pellets may be coated multiple times to increase the dose of drug per pellet and, as described elsewhere herein, talc or magnesium stearate may be added during coating to prevent aggregation of the pellets. The pellets are dried (the coating is dehydrated) to form a solid yet readily water-soluble matrix that comprises the drug.

The pellets are either loaded into capsules using technology that is well-known in the art or are embedded in tablet form. Embedding the pellets within a tablet is performed by compression on a tablet press equipment. Pellets are mixed with tableting ingredients (filler at a minimum, but may contain other ingredients including disintegrants, binders, lubricants, edulcorates, etc.) and compressed at a suitable pressure to achieve the desired shape and size. Shape of the tablet is determined by the die and punch set used in the tableting machine. Size and weight of the tablet is determined by the amount of material added per tablet die and the pressure applied during the compression process.

It is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Representative illustrative methods and materials are herein described; methods and materials similar or equivalent to those described herein can also be used in the practice or testing of the present invention.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual dates of public availability and may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as support for the recitation in the claims of such exclusive terminology as “solely,” “only” and the like in connection with the recitation of claim elements, or use of a “negative” limitations, such as “wherein [a particular feature or element] is absent”, or “except for [a particular feature or element]”, or “wherein [a particular feature or element] is not present (included, etc.) . . . ”.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

The invention is further described by the following non-limiting examples which further illustrate the invention, and are not intended, nor should they be interpreted to, limit the scope of the invention.

### EXAMPLES

### Example 1. LAAM-Loaded Electrospun Nanofiber Buccal Films for Treating Opioid Use Disorder

We developed a highly drug-loaded electrospun film to deliver LAAM and applied it to the buccal mucosa, which allows faster delivery and enhanced bioavailability. The selected formulation (F5) exhibited the fastest drug release profile, with approximately 100 percent released within 30 minutes. The F5 showed adequate mucoadhesive strength with 0.56 Newton. Additionally, the pharmacokinetic study revealed a considerable difference in bioavailability between the selected buccal film formulation and oral solution (3.5-fold higher).

In this study, we describe an alternative strategy for LAAM delivery via the transmucosal mute. We propose that transmucosal delivery will achieve better therapeutic efficacy by bypassing the first-pass hepatic metabolism of LAAM and requiring a low-dose administration. This may significantly reduce its arrhythmic side effects of prolonging cardiac QTc interval and torsade's de pointes.⁹ The long half-life of this new dosage form will also reduce the daily visit required for methadone to 2-3 times per week, which will increase patient compliance and minimize treatment costs. In summary, a LAAM transmucosal dosage form offers the potential to provide an additional safe, effective, and economic treatment for patients who are not adequately maintained with current therapies.

### Materials and Methods

### Materials

Polyvinyl pyrrolidone (Kollidon® 90F, Mw 900000-1200000 g/mol, LOT 77568968E0, CAS number 9003-39-8) and Polyethylene glycol (PEG, Mw 3340 g/mol, LOT GNE30021B) were a gift from BASF, USA. Eudragit® NM 30 D suspension (Lot No. C200362001, CAS number 9010-88-2) and Eudragit® RL100 (Mw±150,000, Lot number B200206513, Cas-No: 33434-24-2) were a gift from Evonik. Propylene glycol (Mw 76.09 g/mol, Lot #SHBN3303, CAS 57-55-6) was purchased from Sigma Aldrich, USA. LAAM (as HCL salt) was obtained from the National Institute of Drug Abuse (NIDA) Drug Supply Program. Rabbit plasma was purchased from Biochemed Services (Virginia, US). All other research reagents used were of LC-MS grade or higher.

### Preparation of Polymer Solution

Polyvinyl pyrrolidone (PVP) and ethyl cellulose (EC) were dissolved in ethanol at 15% (w/w) and 5% (w/w) respectively. 10% (w/w) Eudragit® RL 100 was prepared by dissolving in ethanol-water (v/v) solution and polyethylene glycol (PEG) was dissolved in water at 15% (w/w). To prepare electrospinning fibers, polymer solutions were prepared and mixed with LAAM. The mixed solutions were stirred for 2 hours at room temperature before electrospinning.

### Preparation of Eudragit® NM 30 D Backing Layer

The backing layer was made using a solvent-casting film method. A Eudragit® NM 30 D suspension 30% (w/w) (in water) was poured into a petri dish (7 grams of Eudragit® NM 30D per dish) and dried at room temperature (25° C., humidity 40-50%) for 24 hours. The films were peeled off the dish after the solvent evaporated.

### Preparation of LAAM Electrospun Fiber Films

Drug-polymers solutions were loaded into a 10 mL Becton Dickinson syringe with a 22G needle for electrospinning. The electrospinning process was conducted at 15 kV, a flow rate pf 1000 μL/h, a temperature range of from 25-30° C., and a humidity range of from 20-30%. The material and surface area of the collector was a 420 cm² piece of aluminum foil with. The distance between the needle tip and collector was 15 cm. Electrospinning was performed using a FLUIDNATEK® LE-50 electrospinning machine.

### Scanning Electron Microscope

Nanofiber morphology was measured using a Scanning Electron Microscope (SEM). Images were taken using SEM Hitachi FE-SEM SU-70 operated at 5 kV voltage. An appropriate size (amount) of nanofibers was cut and placed onto a platinum plate for sputtering.

### Drug Release Study

The drug release study was conducted in 100 mL of PBS, pH=6.8 at 37° C. under constant stirring (100 RPM). The release media was collected at predetermined time intervals (5, 10, 15, 20, 25, 30, 40, 50, 60, and 120 minutes) and replaced with the same volume of fresh media. Drug levels in the release media were measured using HPLC-UV (Shimadzu) at 216 nm wavelength. The mobile phase was composed of 30/70 water/acetonitrile with added 0.5% triethylamine and acetic acid to titrate the pH down to 6.4-6.8. The stational phase was a Pursuit 5 C18 column.

### Mucoadhesive Strength

The mucoadhesive strength of the electrospun film was measured using a Bose Electroforce® 3200 series instrument. Each film sample had a length and width of 5 cm and 1.5 cm, respectively, with a contact area of 2 cm. The nanofiber was tested using a loaded cell of 250 g at a dispatch speed of 0.02 mm/min. Mucoadhesive strength was measured by the weight of the load that detached the film from the porcine buccal mucosa. The maximum load (in grams) was measured. The calculation for mucoadhesive strength (in Newtons) is the following:

$$Mucoadhesive strength ( Newtons ) = 0.0098 \times maximum load ( gram force )$$

Drug Remaining after 1-Hour Administration to Rabbits

New Zealand white rabbits of mixed gender (weighing 3.6-4.0 kg) were used in this study. The Institutional Animal Care and Use Committee at Virginia Commonwealth University approved all animal protocols. Rabbits were anesthetized following an intramuscular injection of ketamine (50 mg/kg) and xylazine (5-7 mg/kg), followed by applying buccal films (1 mg/kg) into the buccal pouch. Buccal films were taken after 1-hour administration. The remaining drug was measured by placing buccal film into rabbit buccal mucosa. The concentrations of LAAM and its active metabolites were determined using HPLC-UV.

### Permeability Study

A Franz Diffusion Cell was used to measure permeability. A ˜500 μm porcine buccal mucosa was used as the mucosal barrier. The apical side of the mucosa was facing toward the donor compartment of the Franz diffusion where the fiber film was attached. 0.1- and 5-mL PBS at pH=6.8 was added respectively to the donor and receptor chamber. The solution in the receptor chamber was constantly stirred at 600 rpm, and 400 μL samples were withdrawn and replaced with the same volume of fresh PBS (pH=6.8) at 5, 15, 30, 45, 60, 120, 180, 240, 300, 360 and 420 minutes. All samples were then analyzed via LCMS-MS. The apparent permeability coefficients (Pₐₚₚ) were calculated as follows:

$$Papp = ( \frac{d Q}{dt} ) \times \frac{1}{A \times C}$$

$$where \frac{dQ}{dt} = \frac{ΔConcentration}{Δ Time at Steady State} = Slope ,$$

A is the membrane area of drug permeation, and C is the initial concentration in the donor chamber.

### Pharmacokinetics Study in Rabbits

Twenty-four New Zealand white rabbits of mixed gender (weighing 3.6-4.0 kg) were obtained from ENVIGO and used in this study. Animals were housed in an environmentally controlled room (12:12 h light-dark cycle) with free access to antibiotic-free feed and water at least six days before starting any procedures. All animal protocols were approved by the Institutional Animal Care and Use Committee at the Virginia Commonwealth University. All LAAM buccal films and blank films were prepared one day before administration to animals. Rabbits were anesthetized following an intramuscular injection of ketamine (50 mg/kg) and xylazine (5-7 mg/kg), followed by applying buccal films (1 mg/kg) into the buccal pouch. LAAM oral solutions (in water) were prepared freshly on the day of the study as a control group. An oral solution (1 mg/kg) was administered by an oral syringe (1 mL), followed by an injection of ketamine/xylazine. After anesthesia, a catheter was inserted via the marginal ear vein for blood collection before dosing and at pre-determined time points (30 min before and at 5, 15, 30 min, and 1, 2, 3, 4, 8, 12, 24, 48, 72, 96, and 120 h) following administration. Eppendorf tubes contained EDTA (0.5 M) to prevent blood coagulation. Blood samples were placed on ice immediately following the collection for no longer than 5 min, plasma was separated by centrifugation (3000 g, at 0° C. for 3 min) and stored at −80° C. until analysis. At the endpoint (120 h), all animals were sacrificed. Urine, interest tissues (buccal mucosa, kidney, spleen, liver, gallbladder, muscle, brain, and spinal cord, mesenteric fat, perirenal fat, neck fat, inguinal fat) were harvested and stored at −80° C. until analysis. The buccal mucosa tissues and control in contact with fiber film formulations were collected for histopathological analysis using H&E staining. The concentrations of LAAM and its active metabolites in plasma and tissues were determined by means of LC-MS/MS. Non-compartmental analysis (NCA) was applied to calculate pharmacokinetic parameters derived from plasma concentration-time profiles. The absolute bioavailability was subsequently calculated by comparing respective AUCs, as shown by the following equation:

$$Bioavailability ( F \% ) = \frac{A U {C}_{Buccal} \times {Dose}_{IV}}{A U {C}_{IV} \times {Dose}_{B u c c a l}}$$

### Histology Study of the Buccal Mucosa

After the end-point of the pharmacokinetic study (120 h), the rabbits were euthanized using 100 mg/kg Euthanasia solution (Pentobarbital sodium and phenytoin sodium solution mixture) via IV. The rabbit mucosa was sectioned using surgery blade and stored in 10% formalin solution. After the buccal film application, the rabbits were sacrificed at pre-determined time points (120 h). The buccal mucosa tissue was harvested and fixed in a 10% formalin solution for 24 hours. The tissue was then dehydrated using an ascending series of ethanol solutions and embedded in paraffin. The paraffin-embedded tissue blocks were cut into five μm sections using a microtome and stained with hematoxylin and eosin (H&E) for histological evaluation. The tissue sections were examined under a light microscope for any signs of tissue damage, inflammation, or other histological changes.

### Stability Study

The stability study was conducted by storing the buccal film at different temperatures and humidity levels. The samples were stored at 25° C. and 60% RH for up to three months. At pre-determined time points (0, 1, 2, and 3 months), the samples were removed and analyzed for drug content, appearance, and drug release. Buccal films were manufactured using an electrospinning process to begin the study. The films were then cut into rectangular shapes of approximately one cm² and stored at temperature and humidity conditions: 25° C. and 60% RH. The samples were stored for up to three months, with samples being taken at pre-determined time points (0, 1, 2, 3 and 6 months) for analysis. The stability of the drug was evaluated using several methods. The drug concentration was determined using HPLC-UV. The physical appearance of the buccal films was monitored for any changes in color and texture. Drug release was measured using a USP apparatus 1.

### Results

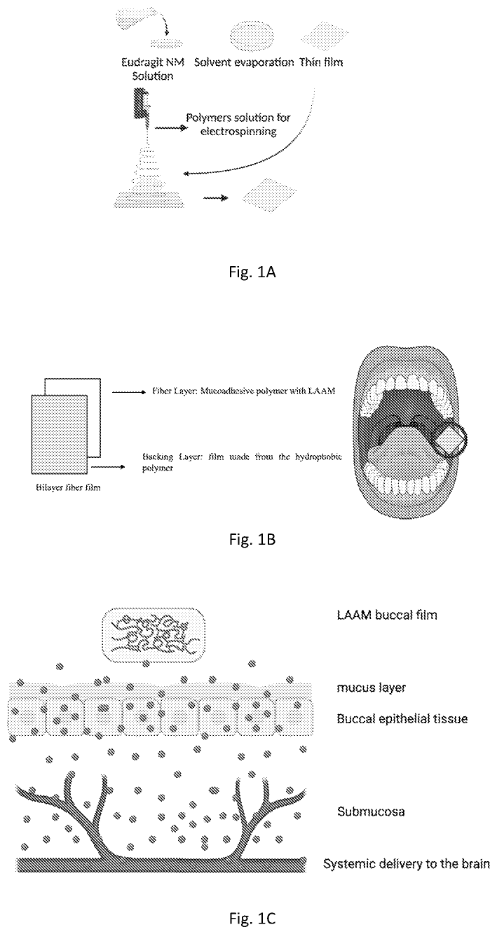

We designed a bilayer fiber film comprising an electrospun fiber layer and a hydrophobic backing layer made using a casting solvent technique (FIG. 1A). When applied to the buccal mucosa (FIG. 1B), the electrospun fiber layer makes direct contact with the mucosa and delivers LAAM thereto, while the backing layer prevents drug release into the oral cavity. Thus, overall, drug release into buccal mucosa is promoted (FIG. 1C). In addition to promoting unidirectional release into buccal mucosa, the backing layer was also introduced for better patient comfort, given that nanofibrous mucoadhesive films dissolve or erode relatively rapidly without the need for removal.

### Preparation and Characterization of Unidirectional Release LAAM Buccal Film

By incorporating the LAAM into the polymer solutions, we were able to produce a highly loaded fiber buccal film. The electrospinning process allows the loading of approximately 20 mg of drug or around 50 percent drug loading per 4.25 cm² buccal film size which is the amount of dose recommended for low or unknown tolerance of the OUD patient. We have developed sets of formulations with polymer combinations, as shown in Table 1. Excipients used for the electrospun fiber layer were selected from the FDA approved list of active ingredients and included mucoadhesive and rate-controlling polymers like ethyl cellulose (EC) and Eudragit® RL 100 and rapidly water soluble and mucoadhesive polymers like polyvinylpyrrolidone (PVP) and polyethylene glycol (PEG). PVP was selected due to its proven record as a safe, non-toxic, non-irritant, biocompatible polymer and hence it is completely safe for usage in pharmaceutical applications. We also added PG as a penetration enhancer to enhance absorption into buccal mucosa. Hydrophobic polymers such as EC and Eudragit® RL 100 were added to enhance buccal film mucoadhesive properties. PEG was incorporated in order to prevent shrinkage of the buccal film.

TABLE 1
Formulation compositions and characterization of LAAM buccal film

| | | Drug release | | In vivo drug | |
|---|---|---|---|---|---|
| | PVP-EC-RL100-PEG-PG- | in 5 minutes | Mucoadhesive | remaining (%) on | |
| | LAAM Composition (% | (% Mean ± | Strength | buccal film after | Pₐₚₚ |
| Code | w/w) | STD) | (Newton) | 1 h administration | (cm/sec) |
| F0 | 23.8-23.8-0-0-2.4-50 | 70.9 ± 2.1 | NA | NA | NA |
| F1 | 38.1-9.5-0-0-2.4-50 | 88.9 ± 0.3 | 0.3 ± 0.01 | 47.36 ± 8.29 | |
| F2 | 38.1-0-9.5-0-2.4-50 | 94.3 ± 3.9 | 1.1 ± 0.22 | 58.02 ± 9.04 | |
| F3 | 38.1-4.75-4.75-0-2.4-50 | 86.8 ± 1.5 | 0.8 ± 0.18 | 51.41 ± 6.79 | |
| F4 | 45.2-0-2.4-0-2.4-50 | 99.9 ± 0.7 | NA | NA | NA |
| F5 | 31.6-0-2.4-13.6-2.4-50 | 96.8 ± 2.0 | 0.56 ± 0.19 | 18.01 ± 3.94 | 1.9 × 10⁻⁵ ± |
| | | | | | 2.5 × 10⁻⁷ |

Cells marked with “NA” indicate that the data is unavailable, as the corresponding experiment was not conducted. Formulation F0 did not meet our requirement for 80% release within 30 minutes, while Formulation F4 fiber buccal film experienced shrinkage after preparation, rendering them unsuitable for further experimentation.

### Fiber Morphology

Bilayer buccal films composed of a mucoadhesive fiber layer and a hydrophobic backing layer (Eudragit® NM 30 D suspension) were successfully fabricated. SEM was used to show the structure or morphology of the fiber buccal film (FIG. 2A). All formulations showed random alignment and exhibited submicron diameters (0.3-0.8 μm) with no apparent defects such as bead formation. Buccal films of five different compositions were manufactured, along with a control film without the drug. F5 became the lead formulation. We observed a coating shape on the F5 SEM image. We further investigated this observation by making the F5 without LAAM and F5 without PEG. F5 without LAAM showed a similar morphology to F5 without PEG which indicates LAAM might react with PEG on the surface of the fiber.

### Drug Release of LAAM

In vitro release concentration of LAAM was measured using reverse phase HPLC. The mean accumulative release rate of LAAM fiber films during 2 h is shown in FIG. 2B. The results indicated that except formulation F0, all other fiber films had a fast drug release rate with 80% of the drug being released within 30 minutes. The fast release is attributed to the rapid dissolution behavior of the submicron-sized water-soluble polymer fibers composed of PVP. F4 and F5, which have the lowest hydrophobic polymer content, (Eudragit® RL 100; 2.4% w/w) showed the fastest drug release (100% and 97% respectively), while F0, F1, F2, and F3 with higher hydrophobic polymer content exhibited a slower drug release of approximately 70, 87, 93, and 86% within 30 minutes.

### Mucoadhesive Test

The mucoadhesive strength (the maximal force required to detach an adhesive surface from a mucous substrate) was measured with the detachment force or tensile method. The obtained mucoadhesive strengths are shown in Table 1. The results showed that F2 had the highest mucoadhesive strength followed by F3, F5, and F1.

Rabbit Study of Drug Remaining 1-Hour after Administration

The concentration of drug remaining in the buccal film 1 hour after administration was determined by assessing the buccal film placed into rabbit buccal mucosa. The mean concentration of LAAM remaining in the buccal film 1-hour after administration is shown in Table 1. We observed that F5 has the lowest drug remaining at 18.01%. F5 showed the fastest drug release (approximately 100% in 10 minutes), adequate mucoadhesive strength, and the lowest amount of drug remaining. These results suggest that F5 is the optimal formulation for drug delivery via buccal films.

### Ex Vivo Permeation Study

The addition of penetration enhancers, in this case, propylene glycol (PG), is essential for transmucosal delivery in order to decrease mucosal barriers. We chose the F5 formulation to undergo an ex vivo permeation study using the Franz diffusion chamber. The results (Table 1) showed that the F5 formulations have a 1.9×10⁻⁵ apparent permeability coefficient. Compared to a model drug having high permeability according to the FDA, such as metoprolol (1.1×10⁻⁵ cm/s), the F5 formulation would also be considered to have high permeability.

### Plasma Pharmacokinetics of LAAM in Rabbits

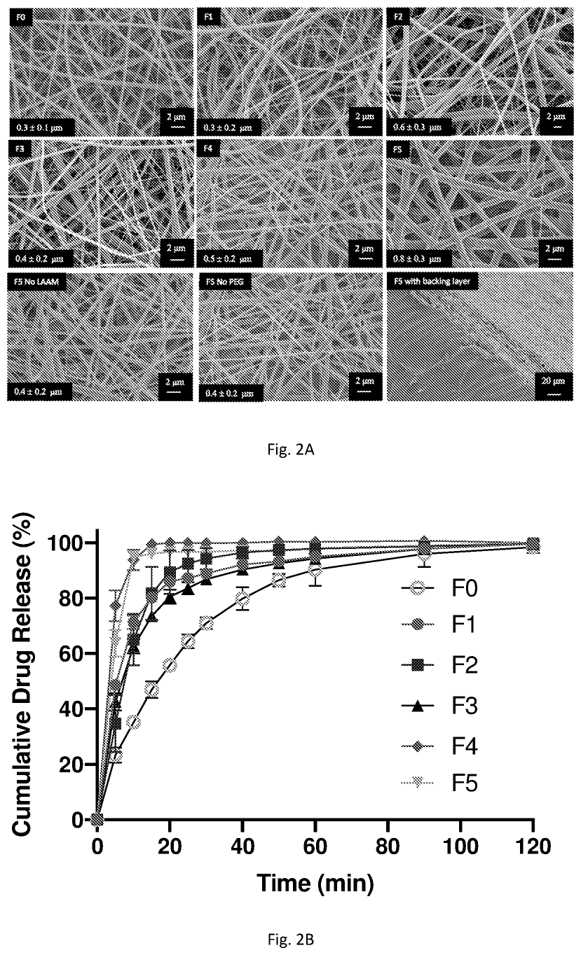

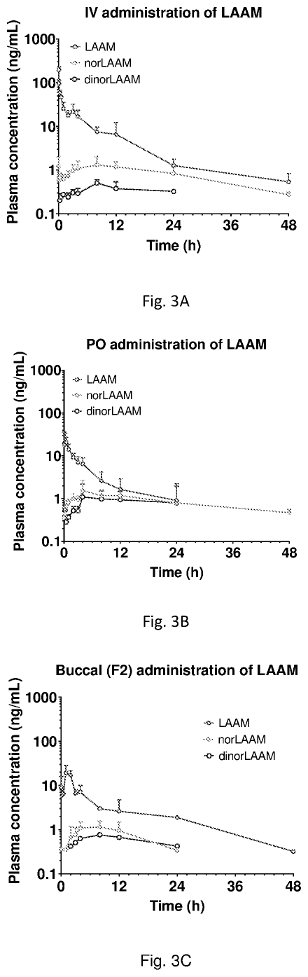

The in vivo pharmacokinetic performances of LAAM were assessed following intravenous, oral and buccal administrations in rabbits (FIG. 3A-D). When LAAM was administered, the plasma samples were analyzed for concentrations of LAAM and its metabolites, nor-LAAM and dinor-LAAM. The pharmacokinetic parameters calculated from these plasma concentration-time profiles are shown in Table 2. There was no statistically significant difference between terminal half-lives (t_{1/2}) of LAAM following different routes of administration and drug formulations. Oral LAAM achieved nearly 20% oral bioavailability. In addition, the bioavailability of LAAM after buccal F2 was slightly higher than those for the oral control, though the differences were not significant. There was a significant difference between buccal F5 v.s. the oral value (3.5-fold higher than oral LAAM) (FIG. 3A-D and Table 2). This was further confirmed via direct comparison with the drug remaining in the buccal F1, 2, 3 (47.4±8.3%, 58.0±9.0%, 51.4±6.8%) and F5 (18.0±3.9%) 1-hour after application, implying that the rapid release of LAAM from the nanofibers was essential for efficient buccal drug delivery. The ratios of nor-LAAM/LAAM AUC_{0→t} were comparable among different routes of administration and formulation. However, the dinor-LAAM/LAAM AUC_{0→t} ratios of oral LAAM were 7.3- and 4.4-fold higher than intravenous and buccal administration, respectively. Additionally, the area under the curve ratios of dinor-LAAM/nor-LAAM was over 2-folder greater following the oral route, compared with other routes. Taken together, these results led to the statistically significant difference between the ratio of (nor-LAAM+dinor-LAAM)/LAAM AUC_{0→t} following oral and other routes, suggesting an extensive presystemic metabolism of LAAM. The time of maximum plasma concentration (tₘₐₓ) was significantly delayed after all buccal administrations compared with oral LAAM (Table 2), suggesting a prolonged oral mucosal absorption. This is likely due to extended residence time in the oral cavity, based on the physicochemical properties of LAAM (e.g., dissolution or solubility).

TABLE 2
Plasma pharmacokinetic parameters of LAAM and its two principal
metabolites following administration of LAAM to rabbits.

| | Formulation | | | |
|---|---|---|---|---|
| | IV | PO | F2 | F5 |
| | Intravenous | Oral | Buccal | Buccal |
| Route of administration | (n = 4) | (n = 5) | (n = 3) | (n = 6) |
| LAAM | | | | |
| AUC_{inf} (h · ng/mL) | 283.5 ± 94.7 | 69.5 ± 10.0 | 122.6 ± 43.4 | 339.0 ± 102.1 |
| AUC_{0→t} (h · ng/mL) | 273.8 ± 91.6 | 65.7 ± 10.1 | 102.8 ± 32.5 | 328.3 ± 105.7 |
| Cₘₐₓ or C₀ (ng/mL) | 287.9 ± 133.1 | 20.7 ± 3.4 | 22.3 ± 7.0 | 52.2 ± 15.3 |
| tₘₐₓ (h) | — | 0.4 ± 0.2 | 1.3 ± 0.6* | 2.3 ± 0.5** |
| t_{1/2} (h) | 11.7 ± 3.3 | 7.1 ± 1.8 | 11.4 ± 7.7 | 10.3 ± 3.1 |
| Vₛₛ (L/kg) | 30.6 ± 6.3 | — | — | — |
| CL (L/h/kg) | 3.1 ± 1.1 | — | — | — |
| F (%) | — | 19.6 ± 2.8 | 30.4 ± 10.0 | 68.7 ± 19.3**** |
| nor-LAAM/LAAM | 0.15 ± 0.14 | 0.31 ± 0.02 | 0.13 ± 0.01 | 0.17 ± 0.12 |
| AUC_{0→t} ratio | | | | |
| dino-rLAAM/LAAM | 0.03 ± 0.02**** | 0.22 ± 0.05 | NA | 0.05 ± 0.02**** |
| AUC_{0→t} ratio | | | | |
| (nor-LAAM + dinor-LAAM)/LAAM | 0.19 ± 0.16** | 0.53 ± 0.05 | NA | 0.22 ± 0.14** |
| AUC_{0→t} ratio | | | | |
| nor-LAAM | | | | |
| AUC_{inf} (h · ng/mL) | 36.7 ± 19.9 | 26.3 ± 4.9 | NA | 61.0 ± 42.1 |
| AUC_{0→t} (h · ng/mL) | 30.1 ± 19.9 | 20.7 ± 4.5 | 13.3 ± 4.1 | 55.2 ± 40.1 |
| Cₘₐₓ (ng/mL) | 1.6 ± 0.6 | 1.8 ± 1.3 | 1.2 ± 0.4 | 2.4 ± 1.3 |
| tₘₐₓ (h) | 4.8 ± 3.9 | 4.1 ± 3.1 | 6.7 ± 2.3 | 8.8 ± 5.0 |
| t_{1/2} (h) | 15.0 ± 5.4 | 10.6 ± 3.9 | NA | 11.4 ± 3.0 |
| dinor-LAAM/nor-LAAM AUC_{0→t} | 0.31 ± 0.17* | 0.72 ± 0.19 | NA | 0.33 ± 0.12** |
| ratio | | | | |
| dinorLAAM | | | | |
| AUC_{inf} (h · ng/mL) | NA | NA | NA | 22.9 ± 19.2 |
| AUC_{0→t} (h · ng/mL) | 7.3 ± 4.3 | 13.1 ± 4.2 | NA | 12.8 ± 8.6 |
| Cₘₐₓ (ng/mL) | 0.4 ± 0.2 | 1.3 ± 1.1 | NA | 0.7 ± 0.4 |
| tₘₐₓ (h) | 7.7 ± 4.5 | 8.8 ± 3.3 | NA | 8.0 ± 0.0 |
| t_{1/2} (h) | NA | NA | NA | 16.2 ± 5.2 |

AUC_{inf}, area under the curve from time zero to infinity; AUC_{0→t}, area under the curve from time zero to the last sampling time point; C₀, concentration extrapolated to time zero; Cₘₐₓ, maximum observed concentration; tₘₐₓ, time to reach peak concentration; t_{1/2}, half-life; Vₛₛ, volume of distribution at steady state; CL, clearance; F (%), bioavailability, calculated by AUC_{inf}.
NA: not available
One-way ANOVA followed by Dunnett's test was used for statistical analysis,
**p < 0.01,
****p < 0.0001 compared to F (%) obtained following oral administration of LAAM.
One-way ANOVA followed by Dunnett's test was used for statistical analysis,
*p < 0.05,
**p < 0.01 and
****p < 0.0001 compared to metabolites/LAAM AUC_{0→t} ratio obtained following oral administration of LAAM.
Unpaired t-test followed by Mann-Whitney test was used for statistical analysis,
*p < 0.05,
**p < 0.01 compared to tₘₐₓ following oral administration of LAAM.

Safety Evaluation after Buccal Film Application on Rabbit Buccal Mucosa

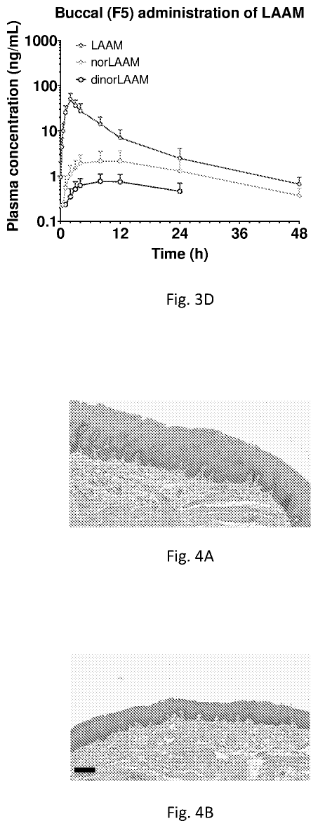

Representative histology images of buccal mucosa at 6 days post-buccal administration using Formulation F5 are shown in FIGS. 4A and B. Formulation F5 maintained epithelium and layer integrity and no inflammation was observed.

### Stability Study

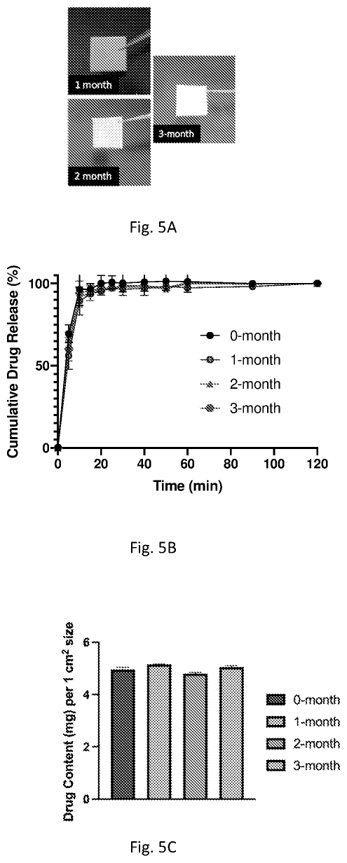

The stability study results indicated that the buccal film was stable under all storage conditions (FIG. 5A-C). The drug content remained within acceptable limits, and the physical appearance of the film did not change significantly during the study period. The study also found that the storage conditions had a minimal effect on the drug's release profile. The drug was found to release approximately 100% within 10 minutes.

### Discussion

To ensure clinical efficacy, buccal films must exhibit sufficient mucoadhesive strength for buccal application and facilitate rapid drug release to minimize application time. We selected nanofiber buccal films due to their large surface area-to-volume ratio, which enables rapid, uniform, and steady drug release profiles. The rapid release is realized using hydrophilic polymers, such as PVP, which exhibit excellent fiber-forming capabilities. However, PVP offers minimal mucoadhesive force. We therefore incorporated hydrophobic polymers such as EC and RL 100 to enhance mucoadhesion. Our target was to achieve at least an 80% drug release within 30 minutes, using FDA guidance for oral solutions as a reference. The preliminary formulation (F0) showed only approximately 60% release within a 30-minute timeframe. Therefore, we increased the hydrophilic polymer ratio to 38.1% w/w in formulations F2 and F3. We also introduced F4, containing a higher PVP ratio (45.2% w/w); however, fiber shrinkage was observed after several days of fabrication. Solution concentration, solvent properties, and post-processing conditions like temperature and humidity can influence electrospun fiber shrinkage. The shrinking behavior, in this case, may be due to the strong interaction between PVP and water molecules, as PVP is highly hydrophilic and readily absorbs water from the environment. When the absorbed water evaporates, the nanofibers can shrink due to polymer chain relaxation and changes in fiber morphology during post-processing.

PEG was incorporated into the formulation (F5) to mitigate fiber shrinkage. PEG is a water-soluble polymer that can be easily incorporated into the electrospinning solution. When subjected to an electric field, PEG molecules migrate to the fiber surface and form a coating, preventing shrinkage and maintaining the fibers' original size and shape.

Drug release study results demonstrated that the polymer composition of the nanofiber matrix significantly influences drug release profiles. More hydrophilic polymers facilitated faster drug release due to increased surface desorption and drug diffusion. F5, with the highest hydrophilic polymer content (31.6% w/w PVP and 13.6% w/w PEG), exhibited approximately 100% drug release within 10 minutes. In contrast, formulations with more hydrophobic components (F1, F2, F3) demonstrated slower drug release rates due to stronger hydrophobic interactions between the drug and the polymer matrix.

We observed that F5 nanofibers had a larger diameter than other formulations. To identify the cause, we created an F5 placebo (PVP-RL100-PEG-PG) and an F5 no-PEG formulation (PVP-RL100-PG-LAAM). SEM images revealed that F5 placebo and F5 no-PEG formulations had smaller diameters (about 0.4 μm) than F5. The increase in fiber diameter could be attributed to drug molecules adsorbing onto the surface of PEG-coated nanofibers or changes in electrospinning parameters upon introducing LAAM to the F5 electrospinning solution.

Mucoadhesive strength for all formulations ranged from 0.3 to 1.1 Newtons. Although no specific recommended value exists for the mucoadhesive strength of buccal films, higher mucoadhesive strength is generally preferred for better retention and prolonged residence time at the application site. Formulation F2, with the highest RL100 ratio (9.5% w/w), exhibited the greatest mucoadhesive strength, followed by F3 and F5 with lower RL100 values (4.75% and 2.4% w/w, respectively). F1, a combination of PVP-EC-PEG, displayed the lowest mucoadhesive strength. This trend suggests that a higher RL100 polymer content increases mucoadhesive strength. Eudragit® RL 100 contains positively charged quaternary ammonium groups, which can interact with the negatively charged sialic acid residues present in the mucus layer. This electrostatic interaction leads to stronger mucus membrane adhesion than ethyl cellulose, which lacks charged functional groups. Additionally, Eudragit® RL 100 is more hydrophilic than ethyl cellulose due to polar functional groups in its structure. This hydrophilicity allows Eudragit® RL 100 to form hydrogen bonds with water molecules in the mucus layer, enhancing its mucoadhesive strength.

We evaluated the in-vivo drug remaining (%) on buccal films after 1-hour administration in rabbits. Formulation F5 showed the lowest amount of remaining drug, likely due to its higher hydrophilic ratio compared to other formulations (F1, F2, F3), which promotes dissolution. Interestingly, formulations F1, F2, and F3 retained approximately 50% of the drug on the buccal mucosa, deviating from the in-vitro drug release results, where 80% of the drug was released within 30 minutes. This difference could be attributed to variations in dissolution media volume between in-vitro and in-vivo conditions. Based on these findings, we concluded that formulation F5 was optimal and selected it for further pharmacokinetic analysis.

Consistent with previous studies in rats, monkeys, and humans, the plasma pharmacokinetic profiles following single bolus intravenous and oral administrations in rabbits showed prolonged exposure to LAAM and its active metabolites nor-LAAM and dinor-LAAM in the blood (up to 48 hours) (FIG. 5). The terminal half-lives (t_{1/2}) of LAAM in rabbits were similar to those reported in humans after intravenous and oral administrations. LAAM's oral bioavailability (Fₒᵣₐₗ %) in humans and rats was approximately 50% and 60%, respectively. However, in this study, poor Fₒᵣₐₗ % of LAAM (nearly 20%) was observed in rabbits. The magnitude of Fₒᵣₐₗ % could be attributed to differences in physiology, CYP450-mediated metabolism, and transport in the gut among various species.

Oral transmucosal drug delivery is an alternative and attractive route of administration to improve systemic exposure of a compound when it undergoes extensive degradation or metabolism in the gastrointestinal tract. Our lead formulation F5, containing a combination of mucoadhesive polymer and permeation enhancer, achieved remarkably high bioavailability (68.7%) of LAAM following buccal administration in rabbits. Additionally, LAAM (log P=4.3) is a weak base with a pKa of 9.87, meaning that in rabbit saliva at pH 9.5±0.3, more LAAM will be in the un-ionized state compared to human saliva (pH 6.2-7.4). This may affect the absorption of LAAM in rabbits compared to humans. Previously published data suggested that nonlinear LAAM kinetics was observed in preclinical and clinical pharmacokinetic studies following single intravenous and oral doses, likely due to the saturation of metabolic processes.

Interestingly, as previously mentioned, a significantly higher % drug unreleased was observed in F2 (≈50%) compared with F5 (≈20%) after 1-hour of in vivo buccal application. Assuming that recalculated doses of LAAM in the films (2 mg for F1, 2, 3 and 3.2 mg for F4) were all ideally absorbed through the oral mucosa to the blood, this suggests that AUC and Cₘₐₓ increases were not dose-proportional in rabbits following buccal administration. For example, the values of Cₘₐₓ and AUC_{0→t} for LAAM of F5 (3.2 mg dose) were approximately 2.3- and 3.2-fold greater, respectively, compared to those of F2 (2 mg dose) (Table 2).

This information informs the selection of the first-in-human dose of F5 for clinical trials.

Histological evaluation of the buccal tissue sections revealed no signs of tissue damage or inflammation. The mucosal layer of the buccal tissue appeared intact, and no inflammation was observed. The absence of histological changes indicates that buccal film application does not cause significant tissue damage.

Throughout the stability study, we measured the drug content and potential degradation over time within the fiber film using HPLC-UV. Our results showed consistent drug content during the three months, suggesting that the fiber film provides a stable drug loading and storage platform. The drug release profile of the stored fiber film was evaluated using dissolution apparatus I. We observed approximately 100% release within 10 minutes, indicating that the polymer-based fiber film is suitable for rapid-release formulations after storage. The physical appearance of the fiber film was monitored over the three months to detect any visible changes in color, transparency, or surface morphology. The material exhibited no significant changes in its appearance, suggesting that it maintains its aesthetic and functional properties during storage. This finding is essential for applications where the appearance of the material is crucial, such as in packaging or consumer products.

In conclusion, this study demonstrates the potential of nanofiber-based buccal films for the rapid release of LAAM. Optimizing the formulation with a combination of hydrophilic and hydrophobic polymers and PEG as a stabilizer, we successfully achieved a high drug release rate and mucoadhesive strength. The F5 formulation, with the highest hydrophilic content, showed the most promising results, providing high bioavailability and minimal remaining drug on the buccal mucosa. Furthermore, the histological evaluation of the buccal tissue sections confirmed the safety and biocompatibility of this formulation.

### Example 2. Novel LAAM Immediate Release Capsule for Treating Opioid Use Disorder

This example describes experiments conducted to optimize (LAAM) dosage forms as effective medications to treat opioid use disorders. In particular, oral tablet and capsule formulations with various drug strengths and an immediate release license profile are described.

### Section 1. Preliminary Experiments Using Diphenhydramine (DPD) as a Model Drug

Under US FDA/CDER guidance, in vitro release experiments were conducted to assess whether coated pellets prepared are capable of releasing the model drug diphenhydramine (DPD), following the immediate release dosage form criterion of 80% dissolution within 30 min. Dose studies were conducted by fully dissolving the coating layers and determining the DPD load per gram of pellets. The optimal DPD formulations were applied to LAAM and LAAM formulations were optimized.

Optimal film-coating conditions were established using a fluid bed coater. Placebo pellets served as an inert bed for coating with drug-polymer mixtures. Drug contents in pellets coated with DPH with four film-forming polymers, Soluplus®, Kollicoat® Protect, Kollicoat® IR and Kollidon® VA64, were determined. 0.1 g of pellets were added with 25 ml of 0.1N HCl which could solve more DPH compared with water and methanol (Table 3). After rotating for 24 h, the samples were centrifuged at 10,000 rpm for 10 minutes. 1 ml supernatant from each sample was removed and filtered via 0.45 um PES, Sterlitech. The drug content was quantitated by HPLC.

TABLE 3
Drug content in different solvent

| Solvents | mg of DPH/g of pellet |
|---|---|
| Water | 5.178 ± 0.082 |
| 0.1N HCl | 6.361 ± 0.120 |
| Methanol | 6.363 ± 0.117 |

Drug content on the pellets was presented by mg DPH/g of pellets. Based on the results, DPH coated with Kollidon® VA64 has the highest drug loading followed by Soluplus® and Kollicoat IR®. Kollicoat® Protect showed the lowest drug loading (Table 4).

TABLE 4
Drug content with different polymers

| Polymer | mg DPH/g of pellets |
|---|---|
| Soluplus ® | 6.498 ± 0.236 |
| Kollicoat ® Protect | 5.704 ± 0.085 |
| Kollicoat ® IR | 6.361 ± 0.120 |
| Kollidon ® VA64 | 9.599 ± 0.093 |

In vitro drug release testing was performed to check whether the drug on the pellets coated with the four different polymer could be released immediately. Under the US FDA/CDER “Dissolution Testing and Acceptance Criteria for Immediate-Release Solid Oral Dosage Form Drug Products Containing High Solubility Drug Substances” guidance, the immediate release dosage form criterion is 80% dissolution within 30 min. The detailed parameters for the dissolution test are shown in Table 5.

TABLE 5
Parameters and criteria for dissolution test for DPH

| Parameters | Criteria | |
|---|---|---|
| Models | USP apparatus 1 with basket | |
| Speed rate | 100 | rpm |
| Release media | 0.1N HCl pH 1.2 | |
| Volume | 500 | mL |
| Temperature | 37 ± 0.5° | C. |
| Weight of pellets | 2 | g |
| Sampling time | 5, 10, 15, 20, 30 min, 1, 2, 3 h | |
| Criteria for immediate release | >80% in 30 min | |
| dosage forms | | |

Based on the release profile, all the products with different polymers are immediately released with 100% cumulative release within 5 mins (data not shown). The pellets coated with Kollicoat® IR had the highest percent of cumulative release. Kollicoat® IR was selected for further formulations because it was easier to formulate, showed instantaneous drug release, and exhibited a proper physical appearance.

### Fluid Bed Coating: Unit Dose Increase

To increase drug content on pellets, methods were developed to increase the ratio of drug to polymer and to double coat the pellets by adding magnesium stearate or talc as a means of priming the surface and reducing stickiness during coating. The ratio of polymer to drug was from 1:1, 1:3 to 1:6. Drug content studies and dissolution tests were performed.

Double-coating using magnesium stearate yielded a 3-fold increase in drug loading by priming the surface and allowing for longer coating processing. Polymer to drug ratio of 1:6 with double coating yielded the highest drug loading. The pellets with 1:6 polymer to drug ratio with double coating also immediately released the drug within 5 mins.

Methods for the quantitative analysis of LAAM were developed using HPLC coupled with a DAD detector. The following chromatographic systems was used:
Column: C18, 4.6×250 mm, 5 μm at ambient temperature
Mobile phase: acetonitrile:water:triethylamine=50:60:0.5, pH adjusted to pH
6.5 by using glacial acetic acid
Flow rate: 1 ml/min
Detector: 218 nm
   LAAM had a retention time of 8.559 min. and the solvent peak appeared at 2-3 min. This method was used for further analyses.

### Fluid-Bed Coating Optimization

Due to the relative low water solubility of LAAM (>15 mg/ml) compared with diphenhydramine hydrochloride (lg/ml), the optimal DPD formulation with 10% (w/w) of drug cannot be applied to LAAM. The formulations of LAAM were thus re-investigated. Using Kollicoat® IR as base polymer initially, a variety of ratios between polymer and LAAM and the concentrations of LAAM were tested and fluid bed coating was performed to obtain an immediate release product with high yield without aggregations. Table 6 shows a summary of coating solution conditions and fluid bed coating parameters in 5 rounds of coating. Coating studies were conducted with 20 g of microcrystalline cellulose (MCC) 700 placebo pellets using a bottom-spray assembly.

TABLE 6
coating solutions and fluid-bed parameters in five rounds of coating processes

| Parameters | Round 1 | Round 2 | Round 3 | Round 4 | Round 5 |
|---|---|---|---|---|---|
| Wt. of LAAM Input (mg) | 450 | 450 | 1350 | 600 | 1500 |
| LAAM Concentration(w/w) | 3% | 2% | 2% | 2% | 2% |
| Kollicoat IR:LAAM | 1:6 | 1:6 | 1:2 | 1:1 | 1:2 |
| Spray on/off cycle | 2.5/0.1 | 2.5/0.1 | 2.5/0.1 | 1.5/1.0 | 2.5/0.1 |
| Peristaltic pump (rpm) | 6 | 6 | 6 | 5 | 9 |
| Coating time (h) | 2.5 | 2 | 7.5 | 7 | 6 |

In the first round, the drug was dissolved after 25 min sonication in 30° C. water bath, but later the drug precipitated out at room temperature. Therefore the 3% w/w drug solution was heated on a magnetic stirrer with controlled temperature when spraying, but the drug still precipitated. In the fourth round, during the coating process, aggregates are easily formed, so 32.4 mg of talc was added when the pellets were coated with 25 ml drug solution.

Dose studies were conducted by fully dissolving the coating layers and determining the drug load per gram of pellets. 0.1 gram of pellets were weighed and dissolved in 25 ml of 0.1 N hydrochloride. The samples were rotated for 24 hours and centrifuged under 10,000 rpm for 10 min. 1 ml supernatant was taken out and passed through 0.45 μm PES filter. HPLC was used for quantitative analysis.

Percent yields after each round of fluid bed coating were calculated. The drug load per gram of pellets and the yield for each round are presented in Table 7. Among different LAAM concentrations, although pellets coated with 3% of LAAM solution have a relatively higher yield, the drug can easily precipitate under room temperature, which may cause a significant problem for the coating process. Among different polymer to drug ratios, the product coated with a 1:2 polymer to drug ratio usually has a higher yield. Therefore, the 2% LAAM solution with 1:2 polymer to drug ratio was used for the fluid bed coating process.

TABLE 7
Drug content and yield in different batch of coating with
different LAAM concentration and polymer to drug ratio

| | Wt. of LAAM | | Yield |
|---|---|---|---|
| Round | Input (mg) | mg LAAM/g pellet | (%) |
| 1 | 450 (3%, 1:6) | 12.2307 ± 0.1076 | 81.35 |
| 2 | 450 (2%, 1:6) | 14.9744 ± 0.3630 | 17.71 |
| 3 | 1350 (2%, 1:2) | 54.5259 ± 0.2539 | 84.54 |
| 4 | 600 (2%, 1:1) | 68.0572 ± 0.6262 | 63.69 |
| 5 | 1500 (2%, 1:2) | 111.2794 ± 21.2902 | 78.72 |

In vitro release experiments were conducted to assess whether coated pellets are capable of releasing LAAM under the US FDA/CDER “Dissolution Testing and Acceptance Criteria for Immediate-Release Solid Oral Dosage Form Drug Products Containing High Solubility Drug Substances” guidance, following the immediate release dosage form criterion of 80% dissolution within 30 min. The detailed parameters for the dissolution test are shown in the following Table 8.

TABLE 8
Parameters and criteria for dissolution test for LAAM

| Parameters | Criteria | |
|---|---|---|
| Models | USP apparatus 1 with basket | |
| Speed rate | 100 | rpm |
| Release media | 0.1N HCl pH 1.2 | |
| Volume | 500 | mL |
| Temperature | 37 ± 0.5° | C. |
| Weight of pellets | 2 | g |
| Sampling time | 5, 10, 15, 20, 30 min, 1, 2, 3 h | |
| Criteria for immediate release | >80% in 30 min | |
| dosage forms | | |

Based on the dissolution curve with the percentage of cumulative release of LAAM according to time, immediate drug release was achieved with 100% LAAM release within 15 min at pH 1.2.

The selection of polymer in the formulation of a 2% LAAM solution with 1:2 polymer to drug ratio was further investigated. Kollicoat® Protect, Sulopuls® and Kollicoat® IR were investigated. The formulation and fluid bed coating parameters are summarized in Table 9.

TABLE 9
Coating solutions and process for 1:2 (w/w) polymer:drug using Kollicoat ®
Protect, Suloplus ® and Kollicoat ® IR

| | Kollicoat ® | | Kollicoat ® |
|---|---|---|---|
| Parameters | Protect | Suloplus ® | IR |
| Wt. of LAAM Input (mg) | 400 | 400 | 400 |
| LAAM Concentration(w/w) | 2% | 2% | 2% |
| Polymer: LAAM | 1:2 | 1:2 | 1:2 |
| Spray on/off cycle | 2.5/0.1 | 2.5/0.1 | 2.5/0.1 |
| Peristaltic pump (rpm) and | 9 rpm for 1.3 h | 9 rpm for 45 min | 9 rpm for 45 min |
| spraying time | 5-7 rpm for 0.5 h | 7 rpm for 15 min | |

### Section 2

In this section, we describe the further development of a novel oral immediate release capsule of LAAM as an alternative to the approved solution formulation, to allow flexible dosing and maintaining similar pharmacokinetic profiles. LAAM solid dispersion-coated pellets were developed with VIVAPUR® MCC Spheres 700 as the core, and a LAAM-carrier coating solution was sprayed onto the core pellets using a fluid bed coater. The LAAM-carrier coating solution was optimized. A Kollicoat® IR-to-LAAM ratio of 1:2 with 2% (w/w) LAAM was selected as the final coating solution formulation based on higher yield and less pellet aggregation during preparation. LAAM coated pellets were filled in size #1 clear gelatin capsules and packaged in blister packs. Capsules with different dosage strengths (5 mg, 10 mg, and 40 mg) were prepared with 100 capsules per dosage strength. Capsule weight variation, content uniformity, and in vitro release tests were conducted for quality assessment. There was no deviation outside ±10% with any weight of a finished capsule's contents and the weight of a finished capsule. All three dosage strengths passed the content uniformity test. The LAAM capsules showed an immediate release profile. The storage stability of the LAAM capsules were evaluated under accelerated conditions and room temperature for 6 months. The weight gain, drug content, and dissolution profile were evaluated at months 1, 2, 3, and 6. The weight gain of capsules stored under accelerated conditions was higher than that of capsules stored in normal conditions. The capsule weights between 1, 2, 3, and 6 months were steady. The results showed that drug contents and immediate release property remained after 6 months of storage. Pharmacokinetic (PK) studies were performed in rabbits. The PK analysis showed that capsules have longer t. as well as higher C. of LAAM compared to oral solution. These capsules have an AUC of LAAM and its metabolites similar to that of oral solutions. Thus, the LAAM capsules have the desired properties for subsequent clinical studies.

### Materials and Methods

### Materials

VIVAPUR® MCC Spheres 700 (CAS #9004-34-6) were gifted. Soluplus®, Kollicoat® is Protect, and Kollicoat® IR were purchased from BASF, USA. LAAM (as the HCL salt) was obtained from the National Institute of Drug Abuse (NIDA) Drug Supply Program. Distilled water was purified in-house (Milli-Q®, Millipore Sigma, Burlington, MA, USA). Hydrochloric acid solution, 6N (Cat #SA56-500) was purchased from Fisher Scientific.

### Preparation of LAAM-Carrier Coating Solution

A LAAM-carrier coating solution was developed by selecting the optimal carriers from carrier candidates and optimizing drug concentration and the drug-to-carrier ratio based on the products' performance. Polymeric carriers including Soluplus®, Kollicoat® Protect, and Kollicoat® IR were selected. Polymeric carriers were compared by mixing with LAAM at a 1:2 ratio of carrier to LAAM and dissolved in water to make a 2% (w/w) LAAM coating solution. The optimal carrier was selected and mixed with LAAM at different ratios of carrier to LAAM (1:1, 1:2, 1:6) and dissolved in water. Different LAAM concentrations varying from 2%-3% (w/w) were achieved.

### Preparation of LAAM-Coated Pellets

The LAAM solid dispersion-coated pellets were produced using a VFC-LAB Micro Fluid Bed Coater using the bottom-spray (Wurster) coating method. VIVAPUR® MCC Spheres 700 were loaded in the product container. LAAM-carrier coating solution (2% w/w, Kollicoat® IR:LAAM=1:2) was sprayed onto the placebo pellets following the experimental conditions below:

$$\begin{matrix}inlet air temperature = 80 ° C . \\ spray on / off cycle = 2.5 / 0.1 .\end{matrix}$$

Peristaltic pump speed was adjusted to maintain a fast rate and avoid pellet aggregation. LAAM solid dispersion-coated pellets with different drug loadings were obtained by increasing the coating time.

### Drug Content of Coated Pellets

Drug loading was quantified by dissolving 0.3 g of drug-coated pellets with 25 ml of 0.1N HCl in 50 ml centrifuge tubes and rotating for 24 hours. Samples were centrifuge at 10,000 rpm for 10 mins. 1 ml of supernatant was passed through a 0.45 um PES filter and prepared for HPLC analysis. Coating efficiency was calculated by dividing the amount of total loaded drug by the amount of drug input.

### HPLC Method

HPLC method for quantitative analysis of LAAM was developed by using a Shimadzu Prominence UFLC system coupled with a diode array detector (DAD). The column was an Agilent 5 HC-C18, 4.6×250 mm (Agilent Technologies). The mobile phase consisted of acetonitrile:water:triethylamine=50:60:0.5 and glacial acetic acid was used to adjust pH to 6.5. The flow rate was 1 ml/min. The injection volume was 20 ul. Chromatograms were recorded under 218 nm.

### In-Vitro Release Testing

Following USP<711> Dissolution guidelines, in vitro release tests were performed by using a dissolution tester (AT Xtend®, Sortax). A USP apparatus I basket was used with the temperature controlled at 37° C. 500 ml of 0.1N HCl receptor medium was added to each vessel and approximately 0.3 g of coated pellets were loaded in the basket. The stirring speed was set at 100 rpm. 1 ml of dissolution media was taken from each sample at 5, 10, 15, 20, 30 mins, 1 hr and 2 hrs. Samples were centrifuged at 15,000 rpm for 10 mins and 900 ul of supernatant were prepared for quantification by HPLC. Drug release profiles were plotted as the percent cumulative amount against time.

### Preparation of LAAM Immediate Release Capsules

LAAM coated pellets were filled in size #1 clear gelatin capsules (Medicsca #3159-09) by using a Jaansun® 100 capsule machine. The capsules were packaged in a Class B 6-month dating small amber blisters (HealthCare Logistics, Circleville, OH) and sealed with foil-backed laminate. Different dosage strengths of capsules were prepared by loading different batches of LAAM-coated pellets with the different drug loading described above.

### LAAM Immediate Release Capsules Quality Control

Weight variation for capsules and content tests were conducted based on USP <1163> Quality Assurance in Pharmaceutical Compounding. Ten capsules were randomly picked form each dosage strength and the weights were recorded. Content uniformity testing is performed based on USP <905> Uniformity of Dosage Units. Ten capsules were randomly picked from each dosage strength. Drug loading was quantified by dissolving pellets from a capsule with 25 ml of 0.1 N HCl and followed the preparation procedure for drug loading discussed above. And drug content was evaluated by HPLC method. Based on USP <905>, % label claim of unit i (Xi) was calculated by equation (1). The mean of % label claim of 10 capsules (X̅) was calculated by equation (2). The sample standard deviation (σ) of % label claim was calculated by equation (3). The acceptance value of weight variation is |100−X̅|+(2.4×σ), which should be less than 15. In vitro release test was performed following USP <711> by using USP apparatus I basket under the condition of 100 rpm, 0.1N HCl at 37° C. as discussed above. One capsule was loaded in the basket.

$$\% label claim ( Xi ) = \frac{amount of drug in unit i}{dose on the label} \times 100 \qquad ( 1 )$$

$$\overline{X} = \frac{{\sum }_{i = 1}^{10} X i}{10} \qquad ( 2 )$$

$$σ = \sqrt{\frac{{\sum }_{i = 1}^{10} {( Xi - \overline{X} )}^{2}}{9}} \qquad ( 3 )$$

### Accelerated Stability Test

The storage stability of the LAAM capsules were assessed under accelerated conditions (40° C.±2° C./75% RH t 5% RH) and normal conditions (room temperature) for 6 months. Storage stability tests were performed at 1, 2, 3 and 6 months. Three capsules from each dosage strength and each storage condition were kept at the beginning of the stability test and weight gains were evaluated at different time points. Drug content of capsules from different dosage strength under different storage conditions were compared. Drug content was quantified by HPLC. In vitro release tests were performed by using a USP apparatus I basket. The water content of the capsules was evaluated by Karl Fischer titration (n=3).

### Pharmacokinetics Study in Rabbits

Twelve New Zealand white rabbits of mixed gender (weighing 3.6-4.0 kg) were obtained from ENVIGO and used in this study. Animals were housed in an environmentally controlled room (12:12 h light-dark cycle) with free access to antibiotic-free feed and water at least six days before starting any procedures. Rabbits were orally dosed with size 4 LAAM capsules (1 mg/kg) using a pill gun, followed by an injection of ketamine/xylazine. LAAM oral solutions (in water) were prepared freshly on the day of the PK study as a control group. An oral solution (1 mg/kg) was administered by an oral syringe (1 mL), followed by an injection of ketamine/xylazine. After anesthesia, the catheter was inserted via the marginal ear vein for blood collection before dosing and at pre-determined time points (30 min before and at 5, 15, 30 min, and 1, 2, 3, 4, 8, 12, 24, 48, 72, 96, and 120 h) following administration. Eppendorf tubes contained EDTA (0.5 M) to prevent blood coagulation. Blood samples were placed on ice immediately following the collection for no longer than 5 min, plasma was separated by centrifugation (3000 g, at 0° C. for 3 min) and stored at −80° C. until analysis. At the endpoint (120 h), all animals were sacrificed. The concentrations of LAAM and its active metabolites in plasma and tissues were determined by means of LC-MS/MS. Non-compartmental analysis (NCA) was applied to calculate pharmacokinetic parameters derived from plasma concentration-time profiles. The relative bioavailability can subsequently be calculated by comparing respective AUCs, as shown by the following equation:

$$Relative bioavailability ( {F}_{rel} \% ) = \frac{A U {C}_{extravasular ( a )} \times {Dose}_{extravasular ( b )}}{A U {C}_{extravasular ( b )} \times {Dose}_{extravasular ( a )}}$$

### Results and Discussion

### Optimization of Coating Solution

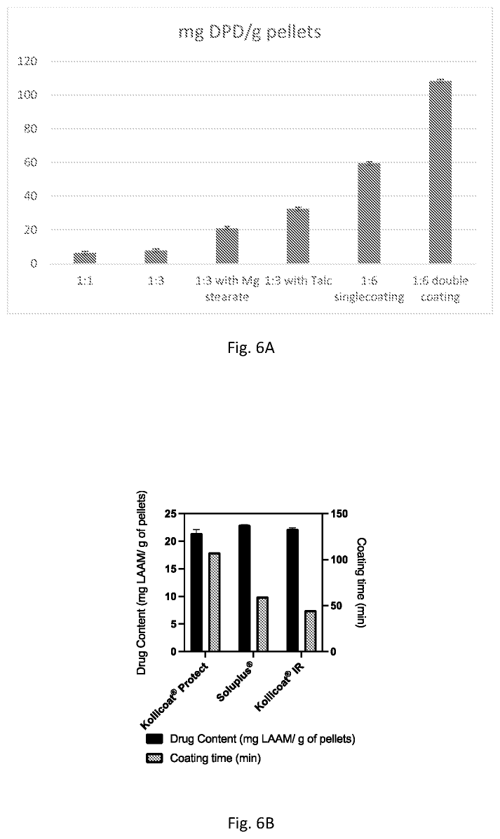

The ideal polymers used in immediate-release film coating should be able to dissolve in aqueous media and facilitate rapid and complete release of API from dosage forms, have the capacity to improve physical and chemical stability of dosage forms, and have no pharmacological activities. Based on these criteria, polymeric carriers including Kollicoat® Protect, Soluplus®, and Kollicoat® IR were selected. Coating solutions were prepared by mixing each individual carrier with LAAM at a 1:2 ratio to make 2% w/w LAAM water solution. Fluid bed coating was processed by spraying the 20 g coating solution on to 20 g MCC sphere 700. The coating process was visually monitored, total coating time was recorded until all the coating solution was used. Total coating time for the formulations using Kollicoat® Protect, Soluplus®, or Kollicoat® IR as a polymer was 108 mins, 60 mins and 45 mins respectively (FIG. 6). Formulation with Kollicoat® IR had the shortest coating time because less aggregation occurred during the coating process and we were able to use a higher peristaltic pump speed with less drying time.

Drug content and production yield of pellets was also tested. The results are shown in Table 10.

TABLE 10
Drug content and production yield of pellets
coated with different polymers

| | Wt. of LAAM | mg LAAM/g | |
|---|---|---|---|
| Sample | Input (mg) | pellet | Yield(%) |
| Kollicoat ® | 405.45 | 21.2519 ± 0.6760 | 104.73 |
| Protect | | | |
| Suloplus ® | 399.45 | 22.9146 ± 0.0593 | 114.92 |
| Kollicoat ®IR | 407.20 | 22.1133 ± 0.1926 | 118.76 |

Further, the drug content and production yield of pellets coated with differing amounts of LAAM were also tested and the results are shown in Table 11.

TABLE 11
Drug content and production yield of pellets
coated with different amount of LAAM

| | Wt. of LAAM | mg LAAM/g | |
|---|---|---|---|
| Sample | Input (mg) | pellet | Yield(%) |
| Kollicoat IR 400 mg | 407.20 | 22.1133 ± 0.1926 | 118.76 |
| Kollicoat IR 2g | 2010.90 | 101.3112 ± 1.0039 | 125.73 |

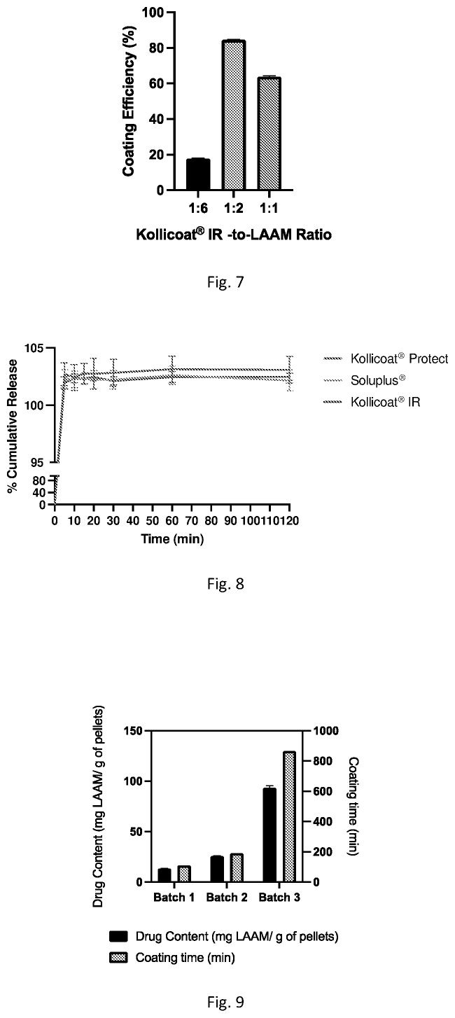

Additional experiments showed that drug contents in formulations with Kollicoat® Protect, Soluplus®, and Kollicoat® IR were 21.49, 22.96, and 22.24 mg LAAM/g of pellets respectively (FIG. 7). Formulations with Soluplus®, and Kollicoat® IR had higher drug content compared with Kollicoat® Protect. In-vitro dissolution tests were performed for these three formulations, and all the formulations were entirely released within 10 mins and thus maintained an immediate release profile (FIG. 8). Considering Soluplus® and Kollicoat® IR formulation had higher drug loading and both maintained an immediate release profile, but Kollicoat® IR achieved the highest drug loading with less coating time, Kollicoat® IR was selected as the polymeric carrier and was used in the following experiments.

Different Kollicoat® IR-to-LAAM ratios in the coating solution were investigated to achieve a high yield of drug loading. 2% (w/w) LAAM solutions were prepared with different Kollicoat® IR-to-drug ratios of 1:1, 1:2 and 1:6. Fluid bed coating was processed by spraying the coating solution onto MCC 700 spheres. The coating efficiency was calculated by dividing real drug content by drug input. In the group with a Kollicoat® IR-to-drug ratio of 1:1, we observed ease of aggregation during the coating process. In the group with a Kollicoat® IR-to-drug ratio of 1:2, no aggregation was observed, and fine particles were formed. In the group with a Kollicoat® IR-to-drug ratio of 1:6, there was no aggregation and fine particles were formed, but we observed a white powder attached to the chamber wall. Drug contents from different groups were analyzed by HPLC and the coating efficiencies were calculated. The group with the Kollicoat® IR-to-drug ratio of 1:2 had the highest yield (FIG. 8). To sum up, the Kollicoat® IR-to-LAAM ratio of 1:2 with 2% (w/w) LAAM was selected as the final coating solution formulation because of ease of process, less coating time, higher drug loading, higher coating efficiency, and immediate-release profile.

### Scale Up Manufacturing of LAAM Solid Dispersion-Coated Pellets by Fluid Bed Coating

For the clinical use of LAAM solution ORLAAM®, a dose of 20 to 40 mg was typically used as a starting treatment, followed by incremental doses of 5 to 10 mg each time until it reached a pharmacokinetic and pharmacodynamic steady state. We aimed at producing three dosage strength of 5 mg, 10 mg, and 40 mg capsules, which correlate to LAAM coated pellets with drug loadings of 13.1, 26.2, and 104.8 mg LAAM/g of pellets respectively, for filling size 1 capsules. The Kollicoat® IR-to-LAAM ratio of 1:2 with 2% (w/w) LAAM was used as the coating solution formulation. LAAM solid dispersion-coated pellets with different drug loadings were obtained by adjusting the coating time. We kept the fluid bed coating parameters constant. We used 2.5/0.5 as the spray on/off cycle and 15 rpm as the peristaltic pump speed. Increasing the coating time led to higher drug loading. The spraying process was manually stopped for longer drying times when aggregation observed. Finally, three batches of 100 g of fine LAAM coated pellets with 13.4, 25.8, and 93.4 mg LAAM/g of pellets were obtained with a coating time of 110, 190, and 865 minutes (FIG. 9). In this study, we did not achieve 104.8 mg LAAM/g of pellets because of time limitations. 104.8 mg LAAM/g of pellets can be achieved if the pellets are coated for a longer time. Coating efficiency was calculated by dividing the amount of total loaded drug by the amount of drug input. We achieved 93.46%, 93.57% and 96.00% coating efficiency for the three batches, respectively. These three batches of pellets with different drug loadings were used in the following study for filling capsules with different dosage strength.

### Preparation Quality Control of LAAM Immediate Release Capsules

Size 1 clear gelatin capsules were filled with different batches of LAAM-coated pellets with the drug loadings discussed above using a Jaansun® 100 capsule machine. The capsules were then packaged in blister packs. Quality assurances are needed to ensure product preparations are produced with quality attributes appropriate to meet the needs of patients and health care professionals. Weight variation for capsules and content tests were conducted based on USP <1163>. The weight of ten capsules and their weight of content were recorded in Table 12. There was no deviation outside ±10% with any weight of a finished capsule's contents and the weight of a finished capsules. This batch of production passed the weight variation test.

TABLE 12
Weight of capsule and the content of each individual
capsule with different dosage strengths

| | Wt. | Wt. | | Wt. | Wt. | | Wt. | Wt. |
|---|---|---|---|---|---|---|---|---|
| | Capsule | Content | | Capsule | Content | | Capsule | Content |
| 5 mg | (mg) | (mg) | 10 mg | (mg) | (mg) | 40 mg | (mg) | (mg) |
| 1 | 454.70 | 379.39 | 1 | 459.16 | 385.49 | 1 | 454.04 | 380.06 |
| 2 | 461.48 | 383.63 | 2 | 457.43 | 380.62 | 2 | 457.54 | 384.68 |
| 3 | 460.29 | 384.01 | 3 | 487.76 | 409.38 | 3 | 461.49 | 385.16 |
| 4 | 465.43 | 387.58 | 4 | 483.64 | 406.48 | 4 | 466.05 | 391.35 |
| 5 | 458.13 | 382.09 | 5 | 475.96 | 397.42 | 5 | 458.60 | 385.57 |
| 6 | 466.14 | 390.07 | 6 | 462.75 | 386.35 | 6 | 465.31 | 389.69 |
| 7 | 467.51 | 392.76 | 7 | 455.34 | 377.95 | 7 | 458.77 | 383.71 |
| 8 | 456.91 | 381.51 | 8 | 452.28 | 375.04 | 8 | 451.75 | 379.54 |
| 9 | 463.07 | 384.33 | 9 | 454.79 | 379.00 | 9 | 466.65 | 390.62 |
| 10 | 464.78 | 388.87 | 10 | 461.28 | 383.73 | 10 | 450.51 | 376.97 |
| Ave.* | 461.84 | 385.42 | Ave. | 465.04 | 388.15 | Ave. | 459.07 | 384.74 |
| SD | 4.29 | 4.23 | SD | 12.72 | 12.11 | SD | 5.83 | 4.88 |

*= average

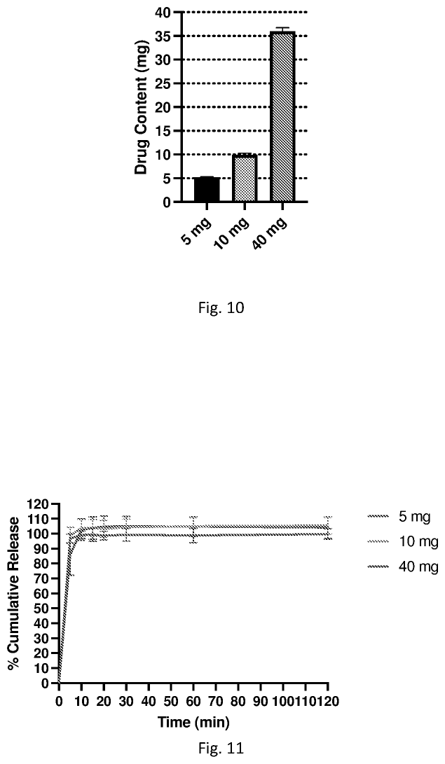

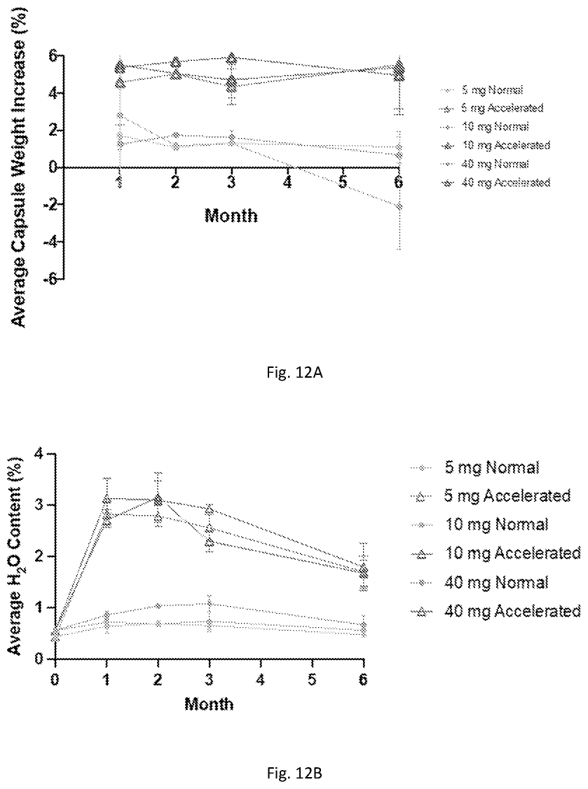

Content uniformity testing was performed based on USP <905>. Based on HPLC analysis, the drug content of the three dosage strengths were 5.17 mg, 9.96 mg, and 35.99 mg (FIG. 10). 40 mg per capsule was not achieved because of lower drug loading on the pellets. 40 mg capsules can be produced using the methods we employed in this study. Based on the calculation, the acceptance value of three dosage strength from low to high were 5.71, 7.74 and 5.70 respectively, which are less than 15. All the capsules of the three dosage strengths passed the content uniformity test. In vitro release tests were performed according to the FDA guideline. Immediate-release dosage form was defined as not less than 80% of the labeled amount of drug is dissolved in 30 minutes. The LAAM capsules maintained the immediate release profile (FIG. 11). All tested capsules got around 100% release within 10 minutes.

### Six-Month Stability Test

Six-month stability tests were conducted at 1, 2, 3, and 6 months. Three capsules were weighted and saved at month 0 and the weights were recorded at different time points. Average capsule weight increases (%) were calculated by dividing the average capsule weight at each time point by the average capsule weight at month 0. During 6 months of storage, there was a 5% weight gain of capsules of all three dosage strengths stored under accelerated condition after one month of storage. The weight gain of capsules under accelerated conditions became stable after the first month. Under normal storage conditions, the average capsule weight increase was lower than that under accelerated conditions and fluctuated during the 6 months of storage. (FIG. 12A) We suspected the weight increase was due to the higher relative humidity of accelerated conditions compared to normal conditions. The high humidity might lead to a higher water content in the capsule. Therefore, we measured the water content of capsules stored under accelerated condition and normal condition during the 6 months. The average H₂O content (%) of capsules stored under accelerated condition increased significantly after 1 month of storage. (FIG. 12 B) The increase of water content might be the major reason for the increase of capsule weight after 1 month storage. However, after 1 month of storage, the water content of capsules stored under accelerated conditions dropped while the weight of the capsules remained the same. Further studies are conducted to investigate the reasons for weight increase of capsules under accelerated conditions.

Drug contents were measured during 6 months of storage. The drug content of capsules of different dosage strengths under different storage conditions were constant for 6 months (FIG. 13).

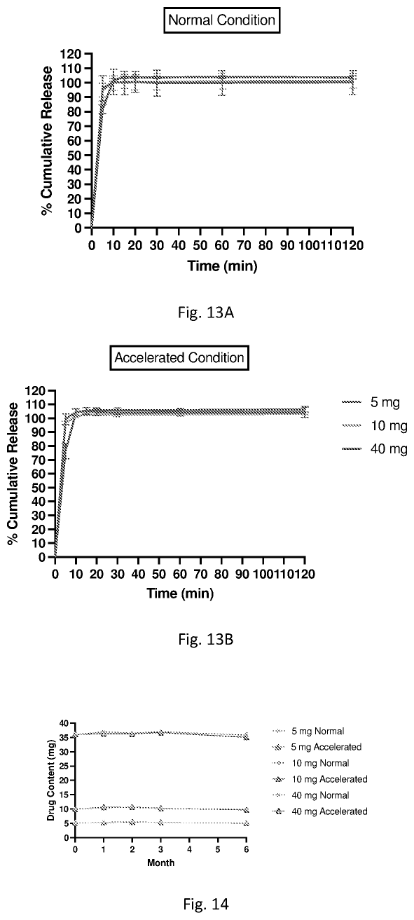

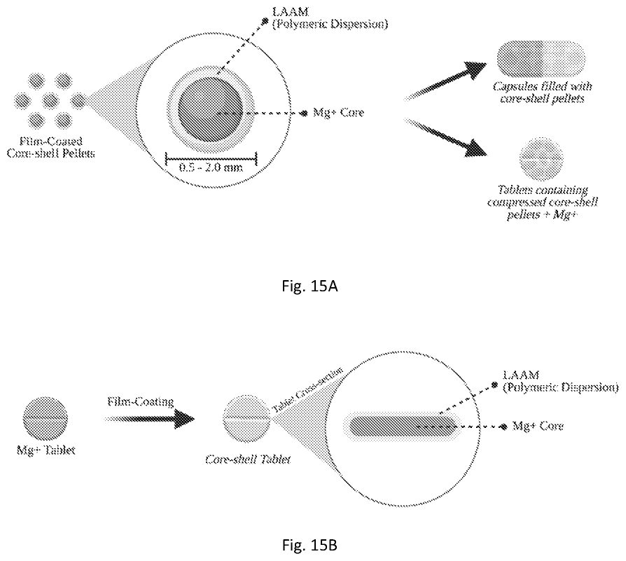

In-vitro release studies were performed for capsules of different dosage strengths under both normal and accelerated conditions after 6 months storage. The LAAM capsules maintained the immediate release profile after 6 months of storage. (FIG. 14)

### Pharmacokinetics Studies

Pharmacokinetic parameters for the capsules and solutions were evaluated in rabbits after a single oral administration by constructing respective plasma concentration time profiles using non-compartmental analysis on each individual animal for each individual analyte (LAAM and its metabolites norLAAM and dinorLAAM). Maximum concentration (Cₘₐₓ), time of maximum concentration (tₘₐₓ), and area under the curve using all observed data points (AUCₐₗₗ) were calculated directly from observed data points and are included for all analytes (Table 13). Compared with the solution form, capsules released their contents slowly and steadily over time. This slow release can help to maintain a high concentration of the drug in the plasma for a longer period. In contrast, solutions in which LAAM is already dissolved in a liquid carrier are designed to be rapidly absorbed by the body. While this can lead to rapid onset of action, it can also result in a more rapid clearance of the drug from the plasma, leading to a lower maximum plasma concentration.

TABLE 13
Summary of pharmacokinetic parameters
of LAAM solution and capsule

| | | Cₘₐₓ | tₘₐₓ | AUCₐₗₗ |
|---|---|---|---|---|
| Formulation | Analyte | (ng/ml) | (min) | (min*ng/ml) |
| Solution | LAAM | 26 | 19 | 6105 |
| | norLAAM | 2.0 | 425 | 1903 |
| | dinorLAAM | 1.2 | 440 | 1279 |
| Capsule | LAAM | 45 | 61 | 8139 |
| | norLAAM | 1.1 | 480 | 1314 |
| | dinorLAAM | 0.7 | 520 | 486 |

### CONCLUSIONS

In this study, we developed a novel LAAM immediate-release capsule for the treatment of opioid use disorder. The capsule was designed to be filled with LAAM coated pellets having a core-shall structure. A LAAM-carrier coating solution was developed by selecting the optimal carriers from carrier candidates and optimizing drug concentration and the drug-to-carrier ratio based on the products' performance. Fluid-bed coating was used for producing large batches of pellets with different drug loadings. Pellets with different drug content were used to make 5 mg, 10 mg and 40 mg LAAM capsules. These three dosage strengths capsules were produced and passed the quality control tests of weight and content variation, and in vitro release profile. Six months storage stability tests were performed. The drug content and immediate-release profile were maintained during storage. These capsules have different but similar Cₘₐₓ AUC values for LAAM and its metabolites, compared to oral solution administration. It has been demonstrated that the developed LAAM capsules are suitable for the treatment of OUD.

### Example 3. Preparation of Tablet Dosage Forms with LAAM-Containing Pellets Embedded Therein

Tablets with LAAM-containing pellets embedded therein are prepared by direct compression using well-established and commercially available tablet press machinery for pharmaceutical purposes. LAAM-containing pellets are blended with a mixture of filler and disintegrant such as microcrystalline cellulose and croscarmellose, yielding approximately 30-60% w/w of the mixture to be compressed. The tableting mixture may contain other ingredients to a smaller extent, including lubricants, glidants, edulcorates, etc. Upon blending, the mixture is fed through a hopper to a tablet press assembled with a set of dies and punches to yield tablets of the desired shape and size (e.g., oval, round, oblong, etc.). Compression occurs at pre-optimized pressures to yield tablets of desired mechanical strength with minimal damage to the integrity and functionality of the pellets.

### Example 4. Novel Approach to Address LAAM QT Interval Prolongation

There has not previously been a study on LAAM-magnesium co-administration to reduce the probability of LAAM-induced QT interval prolongation. We identify the effective extracellular magnesium concentration range and investigate the impact of intracellular magnesium using a human cardiomyocyte-based QT prolongation assay. LAAM/Mg pellets, tablets and/or films are developed (see FIGS. 15A and B) and administered to rabbits orally and the plasma magnesium level is monitored. The effective Mg concentration is determined in vitro and the plasma Mg level in rabbits provides the optimal dosing regimen of magnesium to achieve effective prevention of QT interval prolongation for LAAM.

While the invention has been described in terms of its several exemplary embodiments, those skilled in the art will recognize that the invention can be practiced with modification within the spirit and scope of the appended claims. Accordingly, the present invention should not be limited to the embodiments as described above but should further include all modifications and equivalents thereof within the spirit and scope of the description provided herein.

We claim:

1. A pellet or tablet comprising
a solid core, and
a solid matrix surrounding the solid core, wherein the solid matrix comprises at least one layer comprising a water-soluble polymer and levo-alpha-acetylmethadol (LAAM) or a physiologically active metabolite thereof.

2. The pellet or tablet of claim 1, wherein the solid core is or comprises microcrystalline cellulose.

3. The pellet or tablet of claim 1 or 2, wherein the water-soluble polymer is or comprises a polyethylene glycol-polyvinyl alcohol graft copolymer.

4. The pellet or tablet of claim 3, wherein the polyethylene glycol-polyvinyl alcohol graft copolymer and the LAAM or physiologically active metabolite thereof are present at ratio of 1:2.

5. The pellet or tablet of any of claims 1-4, wherein the solid matrix comprises 2% (w/w) of LAAM or the physiologically active metabolite thereof.

6. The pellet or tablet of any of claims 1-5, wherein the solid matrix comprises a plurality of layers.

7. The pellet or tablet of any of claims 1-6, wherein the physiologically active metabolite is norLAAM or dinorLAMM.

8. The pellet or tablet of any of claims 1-7, wherein the solid core is or comprises Mg or a salt thereof.

9. A water-soluble film comprising electrospun fibers comprising at least one mucoadhesive polymer and LAAM or a physiologically active metabolite thereof and, optionally Mg or a salt thereof.

10. The water-soluble film of claim 9, wherein the at least one mucoadhesive polymer is a copolymer comprising ethyl acrylate, methyl methacrylate and methacrylic acid ester with quaternary ammonium groups.

11. The water-soluble film of claim 9 or 10, further comprising a backing layer that is impermeable to the LAAM or a physiologically active metabolite thereof.

12. The water-soluble film of claim 11, wherein the backing layer comprises a copolymer comprising poly(ethyl acrylate, methyl methacrylate) and polyethylene glycol stearyl ether.

13. The water-soluble film of any of claims 9-12, wherein the electrospun fibers further comprise one or more of a penetration enhancer, a plasticizer, a sweetener and an antioxidant.

14. The water-soluble film of any of claims 9-13, wherein the physiologically active metabolite is norLAAM or dinorLAMM.

15. The water-soluble film of any of claims 9-14, further comprising Mg or a salt thereof.

16. A medicament for the treatment of OUD or pain, comprising
the tablet of claim any of claims 1-8,
a capsule containing a plurality of the pellets of any of claims 1-8,
a tablet comprising a plurality of the pellets of any of claims 1-8 embedded therein, or
the water soluble film of any of claims 9-15.

17. The medicament of claim 16, wherein the levo-alpha-acetylmethadol (LAAM) or the physiologically active metabolite thereof is present in an amount of 5 mg, 10 mg, or 40 mg.

18. An electrospun fiber comprising mucoadhesive polymers and LAAM or a physiologically active metabolite thereof and, optionally, Mg or a salt thereof.

19. A method of treating opioid use disorder (OUD) or pain in a subject in need thereof, comprising,
administering to the subject a therapeutically effective dose of the medicament of claim 16.

20. The method of claim 19, wherein the therapeutically effective dose is from 20 to 160 mg per week.

21. The method of claim 19 or 20, wherein the therapeutically effective dose is administered 1-3 times per week.

22. The method of any of claims 19-21, wherein the step of administering comprises
i) administering the tablet of claim any of claims 1-8, the capsule containing a plurality of the pellets of any of claims 1-8, or the tablet comprising a plurality of the pellets of any of claims 1-8 embedded therein orally; or
ii) administering the water-soluble film of any of claims 9-15 via buccal administration.
